(19) United States
(12) Patent Application Publication
(10) Pub. No.: US 2026/0315808 A1
(43) Pub. Date: Oct. 15, 2026

(54) ADVANCED PACING DELAY DURING ATRIAL FIBRILLATION

(71) Applicant: MEDTRONIC, INC., Minneapolis, MN (US)

(72) Inventors: Robert W. Stadler, Shoreview, MN (US); Xiaohong Zhou, Woodbury, MN (US)

(21) Appl. No.: 18/998,824

(22) PCT Filed: Jul. 12, 2023
(86) PCT No.: PCT/IB2023/057164

§ 371 Date: Jan. 27, 2025

Related U.S. Application Data
(60) Provisional application No. 63/393,072, filed on Jul. 28, 2022.

(51) Int. Cl. A61N 1/368; A61B 5/00; A61B 5/29; A61B 5/361; A61N 1/05; A61N 1/362; A61N 1/37

(52) U.S. Cl. CPC A61N 1/3682; A61B 5/29; A61B 5/361; A61B 5/4836; A61B 5/686; A61N 1/056; A61N 1/3624; A61N 1/3714

(57) ABSTRACT

The present disclosure relates generally to pacing of the cardiac conduction system and/or to the left ventricular septum of a patient, and more particularly, to providing cardiac conducting system and/or left ventricular septal pacing according to an advanced pacing delay during atrial fibrillation. The advanced pacing delay may be determined in various ways either during atrial fibrillation or not during atrial fibrillation.

This application claims the benefit of U.S. Provisional Patent Application Ser. No. 63/393,072, filed 28 Jul. 2022, the entire content of which is incorporated herein by reference.

### TECHNICAL FIELD

The present disclosure relates generally to determination and use of an advanced pacing delay during atrial fibrillation to, for example, ensure capture of the left ventricle.

Implantable medical devices (IMDs), such as cardiac pacemakers or implantable cardioverter defibrillators, deliver therapeutic stimulation to patients' hearts thereby improving the lives of millions of patients living with heart conditions. Conventional pacing techniques involve pacing one or more of the four chambers of a patient's heart 12 as illustrated in FIG. 1, including left atrium (LA) 33, right atrium (RA) 26, left ventricle (LV) 32 and right ventricle (RV) 28. One common conventional therapeutic pacing technique that treats a slow heart rate, referred to as Bradycardia, involves delivering an electrical pulse to a patient's right ventricular tissue. In response to the electrical pulse, both the right and left ventricles contract. However, the heart beat process may be significantly delayed because the pulse travels from the right ventricle through the left ventricle. The electrical pulse passes through the muscle cells that are referred to as myocytes. Myocyte-to-myocyte conduction may be very slow. Delayed electrical pulses can cause the left ventricle to be unable to maintain synchrony with the right ventricle.

Over time, the left ventricle can become significantly inefficient at pumping blood to the body. In some patients, heart failure can develop such that the heart is too weak to pump blood to the body. Heart failure may be a devastating diagnosis since, for example, fifty percent of the heart failure patients have a life expectancy of five years. Another possible cause of heart failure is due to atrial fibrillation, which is an irregular and often very rapid heart rhythm or arrhythmia. During atrial fibrillation, the atria of the heart can beat out of sync with the ventricles of the heart because of the arrythmia of the atria, and this can lead to blood clots in the heart and increase the risk of stroke or heart failure, for example. To avoid the potential development of heart failure, some physicians have considered alternative pacing methods that involve the cardiac conduction system. The cardiac conduction system may be described as being able to quickly conduct electrical pulses (for example, akin to a car driving on a highway), whereas pacing cardiac muscle (myocardial) tissue may more slowly conduct electrical pulses (for example, akin to a car driving on a dirt road).

The cardiac conduction system includes sinoatrial node (SA node) 1, atrial internodal tracts 2, 4, 5 (i.e., anterior internodal 2, middle internodal 4, and posterior internodal 5), atrioventricular node (AV node) 3, His bundle 13 (also known as atrioventricular bundle or bundle of His), and left and right bundle branches 8a, 8b. FIG. 1 also shows the arch of aorta 6 and Bachman's bundle 7. The SA node, located at the junction of the superior vena cava and right atrium, is considered to be the natural pacemaker of the heart since it continuously and repeatedly emits electrical impulses. The electrical impulse spreads through the muscles of right atrium 26 to left atrium 33 to cause synchronous contraction of the atria. Electrical impulses are also carried through atrial internodal tracts to atrioventricular (AV) node 3—the sole connection between the atria and the ventricles. Conduction through the AV nodal tissue takes longer than through the atrial tissue, resulting in a delay between atrial contraction and the start of ventricular contraction. The AV delay, which is the delay between atrial contraction and ventricular contractor, allows the atria to empty blood into the ventricles. Then, the valves between the atria and ventricles close before causing ventricular contraction via branches of the bundle of His. His bundle 13 is located in the membranous atrioventricular septum near the annulus of the tricuspid valve. His bundle 13 splits into left and right bundle branches 8a, 8b and are formed of specialized fibers called “Purkinje fibers” 9. Purkinje fibers 9 may be described as rapidly conducting an action potential down the ventricular septum (VS), spreading the depolarization wavefront quickly through the remaining ventricular myocardium, and producing a coordinated contraction of the ventricular muscle mass.

While cardiac conduction system pacing therapy is increasingly used as an alternative to traditional pacing techniques, cardiac conduction system pacing therapy has not been widely adopted for a variety of reasons. For example, cardiac conduction system pacing electrodes should be positioned within precise target locations (e.g., within about 1 millimeter) of portions or regions of the cardiac conduction system, such as the His bundle, which may be difficult. Additionally, adjustment of cardiac conduction system pacing therapy during delivery of therapy may be challenging. Further, determination of whether the cardiac conduction system pacing therapy is selective (i.e., only pacing the cardiac conduction system) or non-selective (i.e., pacing both the cardiac conduction system and the myocardial tissue) may also be challenging. It is desirable to develop new cardiac conduction system pacing therapy systems, devices, and methods and systems that overcome some of the disadvantages associated with previously-performed cardiac conduction system pacing therapies.

### SUMMARY

This disclosure generally relates to pacing the heart using, for example, cardiac conduction system pacing, left ventricular septal pacing, etc., with an IMD during atrial fibrillation using an advanced pacing delay and/or a selected pacing rate. The left ventricle (LV) may not be effectively captured during atrial fibrillation because, for example, the chaotic activation timing caused by atrial fibrillation may depolarize the ventricles before pacing pulses may be delivered. The advanced pacing delay is representative of a time period or a time period delay from delivery of a pacing pulse (e.g., a left bundle branch pulse) to a ventricular event (e.g., ventricular myocardial depolarization). Using the advanced pacing delay, an IMD may effectively capture and pace the LV during atrial fibrillation. Such effective LV capture may be desirable for patient undergoing cardiac resynchronization therapy (CRT).

In particular, illustrative devices and methods are described herein to provide therapy pacing, that effectively captures the LV during atrial fibrillation. Such effective capture of the LV may be done by pacing the cardiac conduction system, or may be done by pacing left ventricular septal tissue, for example. Pacing the cardiac conduction system may include pacing, for example, the His-Purkinje system, including left bundle branches, right bundle branches, or the His bundle. In particular, illustrative devices and methods are described herein to provide cardiac conduction system pacing therapy that may be able to effectively capture and pace the LV by effectively capturing the left bundle branch (LBB) of the cardiac conduction system. Such cardiac conduction system pacing therapy may be able to determine and adjust the advanced pacing delay based on near-field or far-field signals so as to be able to provide effective cardiac therapy to a patient during atrial fibrillation.

In one or more embodiments, illustrative devices and methods are described herein to provide left ventricular septal pacing (LVSP) that may be able to effectively capture and pace the LV. Such LVSP therapy may be able to determine and adjust the advanced pacing delay based on near-field or far-field signals so as to be able to provide effective cardiac therapy to a patient during atrial fibrillation.

The illustrative devices and methods may be described as utilizing at least a single-chamber device solution for cardiac resynchronization therapy-indicated patients that may include one or more of a standard right atrial lead, a 3830 or 3830 D lead for cardiac conduction system pacing, and a left ventricular lead.

One illustrative implantable medical device may include one or more implantable electrodes to sense and pace a patient's heart. The one or more implantable electrodes comprise a cardiac conduction system electrode positionable proximate a portion of the patient's cardiac conduction system. The device further includes a computing apparatus comprising processing circuitry. The computing apparatus is operably coupled to the one or more implantable electrodes. The computing apparatus is configured to determine an advanced pacing delay to capture the cardiac conduction system. The advanced pacing delay is representative of a time period from delivery of a cardiac conduction system pacing pulse to a ventricular event. The computing apparatus is further configured to initiate delivery of cardiac conduction system pacing during atrial fibrillation using the cardiac conduction system electrode according to the advanced pacing delay.

One illustrative implantable medical device may include one or more implantable electrodes to sense and pace a patient's heart, wherein the one or more implantable electrodes comprise an electrode positionable proximate a left ventricle of the patient's heart. The device may further include a computing apparatus comprising processing circuitry. The computing apparatus is operably coupled to the one or more implantable electrodes. The computing apparatus is configured to determine an advanced pacing delay to capture the ventricular myocardium. The advanced pacing delay is representative of a time period from delivery of a LV septal pacing pulse to a ventricular event. The computing apparatus is further configured to initiate delivery of a LV septal pacing during atrial fibrillation using the electrode according to the advanced pacing delay. To determine the advanced pacing delay, the computing apparatus is further configured to initiate delivery of test LV septal pacing during atrial fibrillation using the electrode according to one or more parameters to capture the ventricular myocardium. The one or more parameters comprises an atrioventricular (AV) delay shorter than an intrinsic AV delay. The computing apparatus is further configured to monitor a far-field electrogram using at least one of the one or more implantable electrodes and one or more surface electrodes during the test LV septal pacing. The computing apparatus is further configured to determine a fiducial point within the far-field electrogram. The computing apparatus is further configured to determine the advanced pacing delay based on the fiducial point. The advanced pacing delay is a delay time period from delivery of a test LV septal pacing pulse of the test LV septal pacing to the fiducial point. The fiducial point is a maximum negative QRS deflection identified within the far-field electrogram.

One illustrative method may include determining an advanced pacing delay to capture a patient's cardiac conduction system. The advanced pacing delay is representative of a time period from delivery of a cardiac conduction system pacing pulse to a ventricular event. The method may further include delivering cardiac conduction system pacing during atrial fibrillation using a cardiac conduction system electrode positioned proximate a portion of a patient's cardiac conduction system according to the advanced pacing delay.

One illustrative method may include determining an advanced pacing delay to capture a patient's left ventricle. The advanced pacing delay is representative of a time period from delivery of a pacing pulse to a ventricular event. The method may include delivering LV septal pacing during atrial fibrillation using an electrode positioned proximate a left ventricle of the patient's heart according to the advanced pacing delay. Determining the advanced pacing delay further comprises delivering test LV septal pacing using the electrode during atrial fibrillation according to one or more parameters to capture the ventricular myocardium. The one or more parameters comprises an atrioventricular (AV) delay shorter than an intrinsic AV delay. Determining the advanced pacing delay further comprises monitoring a far-field electrogram using at least one of one or more implantable electrodes and one or more surface electrodes during the test LV septal pacing. Determining the advanced pacing delay further comprises determining a fiducial point within the far-field electrogram. Determining the advanced pacing delay further comprises determining the advanced pacing delay based on the fiducial point. The advanced pacing delay is a delay time period from delivery of a test LV septal pacing pulse of the test LV septal pacing to the fiducial point. The fiducial point is a maximum negative QRS deflection identified within the far-field electrogram.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a schematic diagram of a heart and conduction system of a patient.

FIG. 2A is a conceptual diagram illustrating an example therapy system (e.g., triple-chamber implantable medical device) that is configured to provide therapy to a heart of patient through a His-bundle or bundle-branch pacing lead and lead placed either in the right ventricle or the right atrium using an implantable medical device (IMD).

FIG. 2B is a conceptual diagram illustrating an example therapy system (e.g., triple-chamber implantable medical device) that is configured to provide therapy to a heart of patient suffering from atrial fibrillation through a His-bundle or bundle-branch pacing lead and lead placed in the left ventricle using an IMD.

FIG. 3A is a schematic diagram illustrating an example His-bundle or bundle-branch pacing lead positioned in bundle of the His via the right atrium in a cross-sectional view of the heart.

FIG. 3B is a schematic diagram illustrating an example of a His-bundle or bundle-branch pacing lead positioned in bundle of the His in a cross-sectional view of the heart using an IMD.

FIG. 3C is a cross-sectional view of a patient's heart implanted with an implantable medical electrical lead to deliver bundle branch pacing.

FIG. 3D is a close-up view of the lead in the patient's heart of FIG. 3C.

FIG. 3E is a conceptual diagram of another example of a medical device system for delivering ventricular cardiac conduction system pacing therapies.

FIG. 3F is a conceptual diagram of yet another example of a medical device system for delivering ventricular cardiac conduction system pacing therapies.

FIG. 4 is a conceptual diagram illustrating an example of a therapy system (e.g., dual chamber implantable medical device) that is configured to provide therapy to a heart of patient through a His-bundle or bundle-branch pacing lead via the right atrium and lead placed in the left ventricle using an IMD.

FIG. 5 is a functional block diagram illustrating an example of a configuration of an implantable medical device of FIG. 2A-B and 3B-4.

FIG. 6 is a block diagram of an illustrative method of determining and using an advanced pacing delay for cardiac conduction system pacing during atrial fibrillation that may be utilized by the devices of FIGS. 2-5.

FIG. 7 is a block diagram of an illustrative method of determining the advanced pacing delay of the method of FIG. 6 for use in cardiac conduction system pacing.

FIG. 8 is a block diagram of another illustrative method of determining the advanced pacing delay of the method of FIG. 6 for use in cardiac conduction system pacing.

FIG. 9 is a depiction of exemplary electrocardiogram and electrogram signals at a plurality of AV delays, illustrating the method of FIG. 8.

FIG. 10 is a block diagram of another illustrative method of determining the advanced pacing delay of the method of FIG. 6 for use in cardiac conduction system pacing.

FIG. 11 is an exemplary graph of QRS width as a function of pacing rate, illustrating the method of FIG. 10.

FIG. 12 is an exemplary graph illustrating an application of the determined advanced pacing delay.

FIG. 13 is an exemplary graph of QRS width as a function of the difference between a pacing rate and an intrinsic rate, which may be applied to the active phase of FIG. 12.

FIG. 14 is a block diagram of an illustrative method of determining and using an advanced pacing delay for LV septal pacing during atrial fibrillation that may be utilized by the devices of FIGS. 2-5.

### DETAILED DESCRIPTION

In the following detailed description of illustrative embodiments, reference is made to the accompanying figures of the drawing which form a part hereof, and in which are shown, by way of illustration, specific embodiments which may be practiced. It is to be understood that other embodiments may be utilized, and structural changes may be made without departing from (e.g., still falling within) the scope of the disclosure presented hereby.

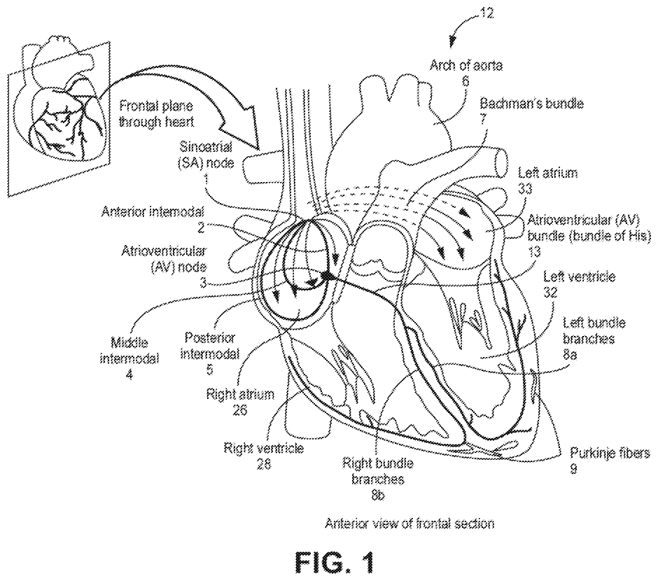

Illustrative systems, devices, and methods shall be described with reference to FIGS. 1-14. It will be apparent to one skilled in the art that elements or processes from one embodiment may be used in combination with elements or processes of the other embodiments, and that the possible embodiments of such systems, devices, and methods using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

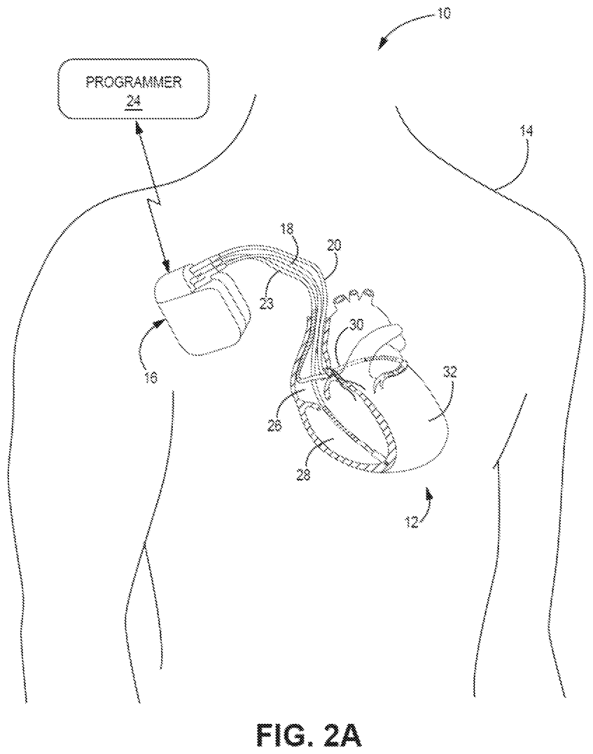

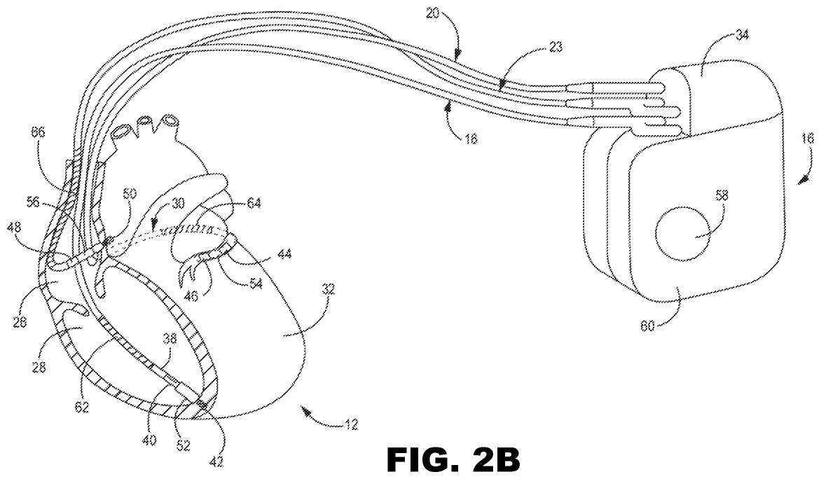

FIG. 1 is a schematic diagram of heart 12. FIGS. 2A-2B are conceptual diagrams illustrating one example therapy system 10 that may be used to provide therapy to heart 12 of patient 14. Patient 14 ordinarily, but not necessarily, will be a human. Therapy system 10 includes IMD 16, which is coupled to leads 18, 20, 23 and programmer 24. IMD 16 may be, for example, an implantable pacemaker, cardioverter, and/or defibrillator that provides electrical signals to heart 12 via electrodes coupled to one or more of leads 18, 20, 23. Further non-limiting examples of IMD 16 include: a pacemaker with a medical lead, an implantable cardioverter-defibrillator (ICD), an intracardiac device, a leadless pacing device (LPD), a subcutaneous ICD (S-ICD), and a subcutaneous medical device (e.g., nerve stimulator, inserted monitoring device, etc.).

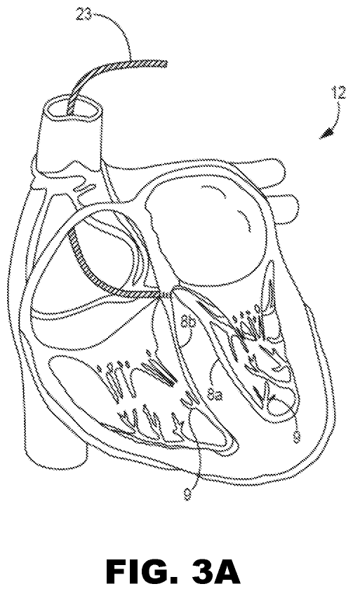

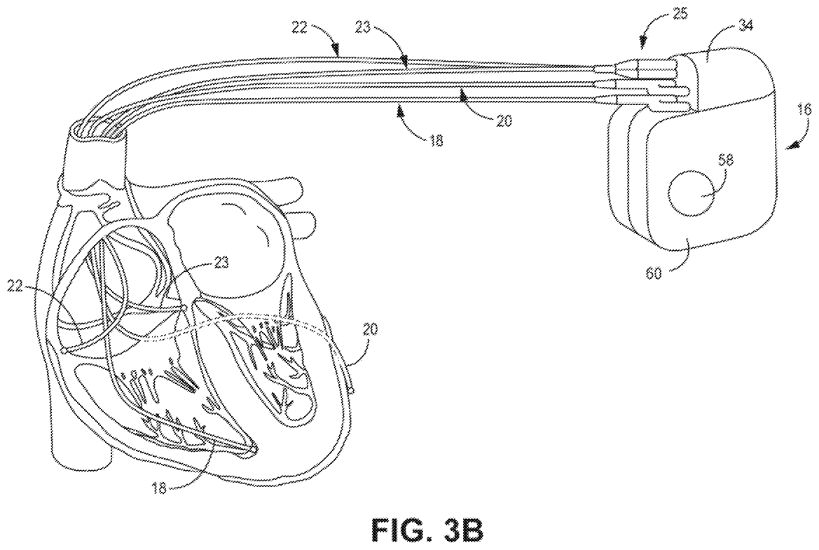

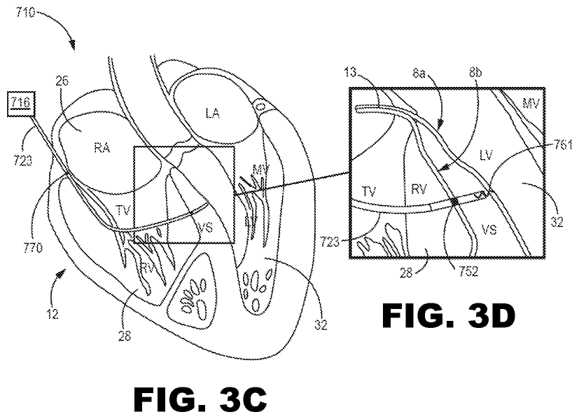

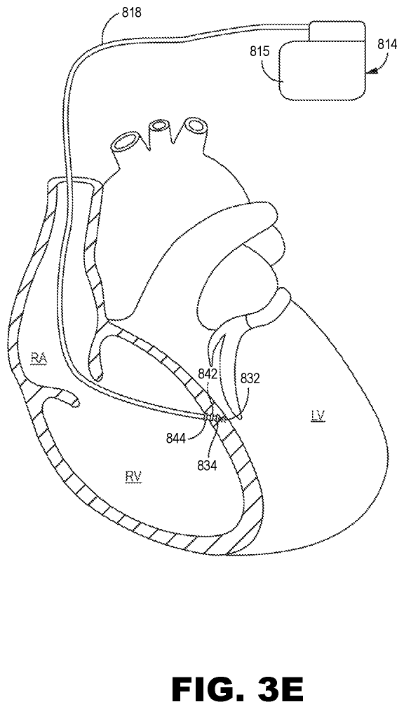

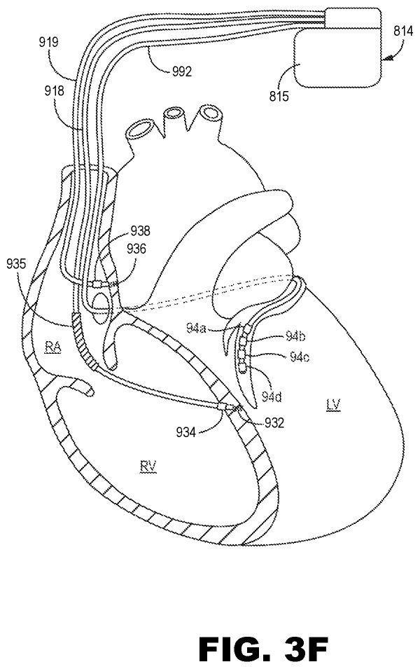

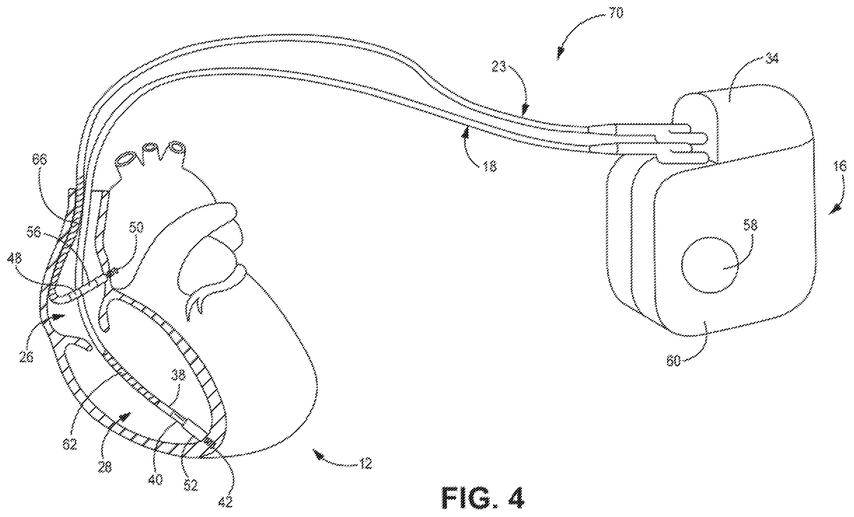

Leads 18, 20, 23 extend into heart 12 of patient 14 to sense electrical activity of heart 12 and/or deliver electrical stimulation to heart 12. In the example shown in FIG. 2A, right ventricular (RV) lead 18 extends through one or more veins (not shown), the superior vena cava (not shown), and right atrium (RA) 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Cardiac conduction system pacing therapy lead 23 (e.g., left bundle branch pacing lead, right bundle branch pacing lead, His-bundle pacing lead, etc.) extends through one or more veins and the vena cava, and into the right atrium 26 of heart 12 to pace the cardiac conduction system (e.g., triangle of Koch, septal wall, left bundle branch, right bundle branch, the His bundle, etc.). In some embodiments, the cardiac conduction system pacing therapy lead 23 may be positioned within about 1 millimeter of the triangle of Koch, septal wall, His bundle, or one or both bundle branches. In one or more embodiments, the cardiac conduction system therapy lead may be further positioned, or located, through the tricuspid valve into the right ventricle and implanted in the interventricular septum, e.g., about 1 to 2 centimeters in an apical direction as will be described further herein with reference to FIGS. 3C-3F.

As used herein, cardiac conduction system pacing therapy refers to any pacing therapy configured to deliver pacing therapy (e.g., pacing pulses) to the cardiac conduction system including, e.g., the His bundle, left bundle branch, right bundle branch, etc. As used herein, the term “activation” refers to a sensed or paced event. For example, an atrial activation may refer to an atrial sense or event (As) or an atrial pace or artifact of atrial pacing (Ap). Similarly, a ventricular activation may refer to a ventricular sense or event (Vs) or a ventricular pace or artifact of ventricular pacing (Vp), which may be described as ventricular stimulation pulses. In some embodiments, activation interval can be detected from As or Ap to Vs or Vp, as well as Vp to Vs. In particular, activation intervals may include a pacing (Ap or Vp) to ventricular interval (LV or RV sense) or an atrial-sensing (As) to ventricular-sensing interval (LV or RV sense).

IMDs may be described as delivering one or both of conventional pacing therapy and cardiac conduction system pacing therapy. Conventional, or traditional, pacing therapy may be described as delivering pacing pulses into myocardial tissue that is not part of the cardiac conduction system of the patient's heart such that, e.g., the pacing pulses trigger electrical activation that propagates primarily from one myocardial cell to another myocardial cell (also referred to as “cell-to-cell”) as opposed to propagating within the cardiac conduction system prior to the myocardial tissue. For instance, conventional pacing therapy may deliver pacing pulses directly into the muscular heart tissue that is to be depolarized to provide the contraction of the heart. For example, conventional left ventricular pacing therapy may utilize a left ventricular (LV) coronary sinus lead that is implanted so as to extend through one or more veins, the vena cava, the right atrium, and into the coronary sinus to a region adjacent to the free wall of the left ventricle of the heart so as to deliver pacing pulses to the myocardial tissue of the free wall of the left ventricle.

One example of a cardiac conduction system pacing therapy lead 23 (e.g., a His lead) can be the SELECTSURE™ 3830. A description of the SELECTSURE™ 3830 is found in the Medtronic model SELECTSURE™ 3830 manual (2013). The SELECTSURE™ 3830 includes two or more conductors with or without lumens.

An exemplary left ventricular lead with a set of spaced apart electrodes is shown in US Pat. Pub. No. WO 2019/104174 A1, filed on May 4, 2012, by Ghosh et al., commonly assigned by the assignee of the present disclosure, the disclosure of which is incorporated by reference in its entirety herein. Exemplary electrodes on leads to form pacing vectors are shown and described in U.S. Pat. No. 8,355,784 B2, US 8,96S,5G7, and U.S. Pat. No. 8,126,546, all of which are incorporated by reference and can implement features of the disclosure.

Additionally, pacing therapy leads 18, 20, 23 may be utilized to deliver left ventricle or left ventricular septal pacing to the ventricular septal wall. At least one of pacing therapy leads 18, 20, 23 may extend through one or more veins, the vena cava, right atrium 26, and into the coronary sinus 30 to a region adjacent to the septal wall of left ventricle 32 of heart 12.

Illustrative cardiac conduction system pacing therapy may be described in, for example, U.S. Pat. App. Pub. No. 2019/0111270 A1 entitled “His Bundle and Bundle Branch Pacing Adjustment” published on Apr. 18, 2019. Illustrative left ventricular septal pacing may be described in, for example, U.S. patent application Ser. No. 16/521,000 entitled “AV Synchronous Septal Pacing” filed on Jul. 24, 2019.

An elongated conductor of the lead may extend through a hermetic feedthrough assembly, and within an insulative tubular member of the lead, and may electrically couple an electrical pulse generator (contained within housing) to the helical tip electrode, or cardiac conduction system electrode, of the cardiac conduction system pacing therapy lead 23. The conductor may be formed by one or more electrically conductive wires, for example, MP35N alloy known to those skilled in the art, in a coiled or cabled configuration, and the insulative tubular member may be any suitable medical grade polymer, for example, polyurethane, silicone rubber, or a blend thereof. According to an illustrative embodiment, the flexible lead body extends a pre-specified length (e.g., about 10 centimeters (cm) to about 20 cm, or about 15 to 20 cm) from a proximal end of housing to the other end. The lead body is less than about 7 French (FR) but typically in the range of about 3 to 4 FR in size. In one or more embodiments, about 2 to about 3 FR size lead body is employed.

Cardiac conduction system pacing therapy can be performed by other leads. Another illustrative lead, including two or more pacing electrodes, can be used to deliver multisite pacing pulses to the cardiac conduction system. Multisite pacing can be delivered simultaneously or sequentially, as described and shown by U.S. Patent Application Publication No. 2016/0339248, filed on Apr. 21, 2016, entitled EFFICIENT DELIVERY OF MULTI-SITE PACING, the disclosure of which is incorporated by reference in its entirety.

Since the electrodes in multi-site or multi-point stimulation may be in close proximity to each other, it may be important to detect and verify effective capture of individual electrodes during delivery of such therapy. Delivering multisite pacing pulses may include delivering pacing pulses to a first tissue site and a second tissue site through first and second pacing electrodes, respectively, all of which may occur within the same cardiac cycle.

In particular, a lead configured to perform multi-site pacing, which is different than LV coronary sinus lead 20, can be placed in the ventricular septum with the first (distal) electrode positioned towards the left side of the ventricular septal wall for left bundle branch pacing and with the second electrode (proximal) towards the right side of the septal wall for pacing the right bundle branch. An interelectrode distance may be defined as the distance between the first and second electrodes, or the distance that the electrodes are apart. In some embodiments, the interelectrode distance is at least about 3, 4, 5, 6, 7, or 8 millimeters (mm). In some embodiments, the interelectrode distance is at most about 15, 14, 13, 12, 11, or 10 mm. For example, the interelectrode distance may be in a range from about 6 to 12 mm apart. Once the pacing is delivered, both the left bundle branch and the right bundle branch may be stimulated such that both ventricles are naturally or near-naturally synchronized. In contrast, in traditional CRT, the ventricles may be described as not naturally synchronized.

A single lead, including two (or more) pacing electrodes (e.g., cathodes) may deliver cathode pacing outputs at two separate locations (e.g., left and right bundle branches), so both bundle branches can be excited at the same time.

Cardiac conduction system pacing may include at least one of His bundle or left or right bundle branch pacing. Bundle branch pacing may bypass the pathological region and may have a low and stable pacing threshold. In some embodiments, only one bundle branch may be paced by using pacing leads. In further embodiments, both bundle branches may be paced at the same time (e.g., dual bundle branch pacing), which may mimic intrinsic activation propagation via the His bundle-Purkinje conduction system, e.g., paced activation propagates via both bundle branches to both ventricles for synchronized contraction. Traditional His bundle pacing, on the other hand, typically paces the His bundle proximal to the bundle branches. In some embodiments, IMD 16 may include one, two, or more electrodes located in one or more bundle branches configured for bundle branch pacing. In some embodiments, IMD 16 may be an intracardiac pacemaker or leadless pacing device (LPD).

As used herein, “leadless” refers to a device being free of a lead extending out of patient's heart 12. In other words, a leadless device may have a lead that does not extend from outside of the patient's heart to inside of the patient's heart. Some leadless devices may be introduced through a vein, but once implanted, the devices are free of, or may not include, any transvenous lead and may be configured to provide cardiac therapy without using any transvenous lead. In one or more embodiments, an LPD for bundle pacing does not use a lead to operably connect to an electrode disposed proximate to the septum when a housing of the device is positioned in the atrium. A leadless electrode may be leadlessly coupled to the housing of the medical device without using a lead between the electrode and the housing.

IMD 16 may sense electrical signals attendant to the depolarization and repolarization of heart 12 via electrodes (not shown in FIGS. 2A-B) coupled to at least one of leads 18, 20, 23. In some examples, IMD 16 provides pacing pulses to heart 12 based on the electrical signals sensed within heart 12. The configurations of electrodes used by IMD 16 for sensing and pacing may be unipolar or bipolar. IMD 16 may also provide defibrillation therapy and/or cardioversion therapy via electrodes located on at least one of leads 18, 20, 23. IMD 16 may detect atrial arrhythmias of heart 12, such as atrial fibrillation of atria 26 and 33, and may deliver defibrillation therapy to heart 12 in the form of electrical pulses. Also, IMD 16 may detect ventricular arrhythmias of heart 12, such as ventricular fibrillation of ventricles 28 and 32, and may deliver defibrillation therapy to heart 12 in the form of electrical pulses. In some examples, IMD 16 may be programmed to deliver a progression of therapies, e.g., pulses with increasing energy levels, until a fibrillation of heart 12 is stopped. IMD 16 may detect fibrillation employing one or more fibrillation detection techniques known in the art.

In some examples, programmer 24 (FIG. 1) may be a handheld computing device or a computer workstation or a mobile phone. Programmer 24 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some embodiments, a display of programmer 24 may include a touch screen display, and a user may interact with programmer 24 via the display. Through the graphical user interface on programmer 24, a user may select one or more optimized parameters.

Additionally, various pacing settings may be adjusted, or configured, based on various sensed signals. For example, various near-field and far-field signals may be sensed by one or more electrodes of the IMD 16 and/or other devices operatively coupled thereto. For example, Vp to QRS end or offset within a near-field or far-field signal may be used to adjust or configure the AV delay of cardiac conduction system pacing therapy. Further, for example, QRS within a near-field or far-field signal may be used to adjust or configure the VV delay between cardiac conduction system pacing therapy and traditional left ventricular pacing therapy. QRS duration is the time from which the Q wave is detected until the S wave ends.

Still further, near-field or far-field signals may be used to adjust or configure the advanced pacing delay. Each of the following exemplary embodiments will be discussed herein in further detail. For example, left bundle branch electrograms or electrocardiograms may be monitored at a plurality of AV delays or at a plurality of pacing rates, and the electrical activity illustrating earliest QRS deflection can be used to determine the advanced pacing delay. Also for example, the left bundle branch electrocardiogram or electrogram illustrating the AV delay associated with fusion pacing and the left bundle branch electrocardiogram or electrogram illustrating the shortest AV delay associated with ineffective LV capture can both be used to determine the advanced pacing delay, as described below. Also for example, the left ventricular septal electrocardiogram or electrogram illustrating the AV delay associated with a maximum negative QRS deflection can be used to determine the advanced pacing delay for LVSP.

Effective fusion may be described as synchronizing the timing of the LV pacing with the earliest activation on the RV chamber. For example, in a fusion pacing configuration, a medical device may deliver one or more fusion pacing pulses to a later-contracting left ventricle (LV) in order to pre-excite the LV and synchronize the depolarization of the LV with the depolarization of the earlier contracting right ventricle (RV). The ventricular activation of the LV may “fuse” (or “merge”) with the ventricular activation of the RV that is attributable to intrinsic conduction of the heart. In this way, the intrinsic and pacing-induced excitation wave fronts may fuse together such that the depolarization of the LV is resynchronized with the depolarization of the RV.

Still further, a left bundle branch electrocardiogram following a post-blanking time period after ventricular pacing may be analyzed to determine whether cardiac conduction system pacing therapy is selective or non-selective.

Thus, near field and/or far-field electrical signals may be used to determine pacing rates resulting in specific QRS morphologies. For example, the far-field electrical signals may be sensed in a far-field electrogram (EGM) monitored by IMD 16 and a corresponding lead or a separate device, such as a subcutaneously implanted device.

As used herein, the term “far-field” electrical signal refers to the result of measuring cardiac activity using a sensor, or electrode, positioned outside of an area of interest. For example, an ECG signal measured from an electrode positioned outside of the patient's heart is one example of a far-field electrical signal of the patient's heart. As another example, a far-field electrical signal representing electrical activity of a chamber of the patient's heart may be measured from a sensor, or electrode, positioned in an adjacent chamber.

As used herein, the term “near-field” electrical signal refers to the result of measuring cardiac activity using a sensor, or electrode, positioned near an area of interest. For example, an EGM signal measured from an electrode positioned on the left side of the patient's ventricular septum is one example of a near-field electrical signal of the patient's LV.

R-wave timing is the time in which QRS is detected. Typically, R-wave timing includes using the maximal first derivative of an R-wave upstroke (or the time of the maximal R-wave value). R-wave timing is also used in the device marker channel to indicate the time of the R-wave or the time of ventricular activation.

Pacing-RV sensing or pacing-LV sensing (e.g., pacing-to-RV sensing or pacing-to-LV sensing) is the time interval from the pacing (or pacing artifact) to the time of RV or LV sensing. For example, if pacing-RV sensing is much longer than pacing-LV sensing, this may indicate that the LV activation is occurring much earlier than RV activation (so pacing-RV sensing is longer), then RV pacing may be delivered in synchronization with bundle pacing, so RV and LV activation can occur approximately at the same time.

A user, such as a physician, technician, or other clinician, may interact with programmer 24 to communicate with IMD 16. For example, the user may interact with programmer 24 to retrieve physiological or diagnostic information from IMD 16. One illustrative IMD 16 is described in the Medtronic AMPLIA MRI™ CRT-D SURESCAN™ DTMB2D1 manual, which is incorporated by reference in its entirety. A user may also interact with programmer 24 to program IMD 16, e.g., select values for operational parameters of the IMD.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

FIG. 2B is a conceptual diagram illustrating IMD 16 and leads 18, 20, 23 of therapy system 10 in greater detail. The triple chamber IMD 16 may be used for cardiac rhythm therapy and defibrillation or cardioversion therapy (CRT-D). Leads 18, 20, 23 may be electrically coupled to a stimulation generator, a sensing module, or other modules of IMD 16 via connector block 34. In some examples, proximal ends of leads 18, 20, 23 may include electrical contacts that electrically couple to respective electrical contacts within connector block 34. In addition, in some examples, leads 18, 20, 23 may be mechanically coupled to connector block 34 with the aid of set screws, connection pins, or another suitable mechanical coupling mechanism.

Each of the leads 18, 20, 23 includes an elongated, insulative lead body, which may carry any number of concentric coiled conductors separated from one another by tubular, insulative sheaths. In the illustrated example, an optional pressure sensor 38 and bipolar electrodes 40 and 42 are located proximate to a distal end of lead 18. In addition, bipolar electrodes 44 and 46 are located proximate to a distal end of lead 20 and bipolar electrodes 48 and 50 are located proximate to a distal end of lead 23. In FIG. 2B, pressure sensor 38 is disposed in right ventricle 28. Pressure sensor 38 may respond to an absolute pressure inside right ventricle 28, and may be, for example, a capacitive or piezoelectric absolute pressure sensor. In other examples, pressure sensor 38 may be positioned within other regions of heart 12 and may monitor pressure within one or more of the other regions of heart 12, or pressure sensor 38 may be positioned elsewhere within or proximate to the cardiovascular system of patient 14 to monitor cardiovascular pressure associated with mechanical contraction of the heart. Optionally, a pressure sensor in the pulmonary artery can be used that is in communication with IMD 16.

Electrodes 40, 44 and 48 may take the form of ring electrodes, and electrodes 42, 46 and 50 may take the form of extendable and/or fixed helix tip electrodes mounted within insulative electrode heads 52, 54 and 56, respectively. Each of electrodes 40, 42, 44, 46, 48 and 50 may be electrically coupled to a respective one of the coiled conductors within the lead body of its associated lead 18, 20, 23, and thereby coupled to respective ones of the electrical contacts on the proximal end of leads 18, 20 23.

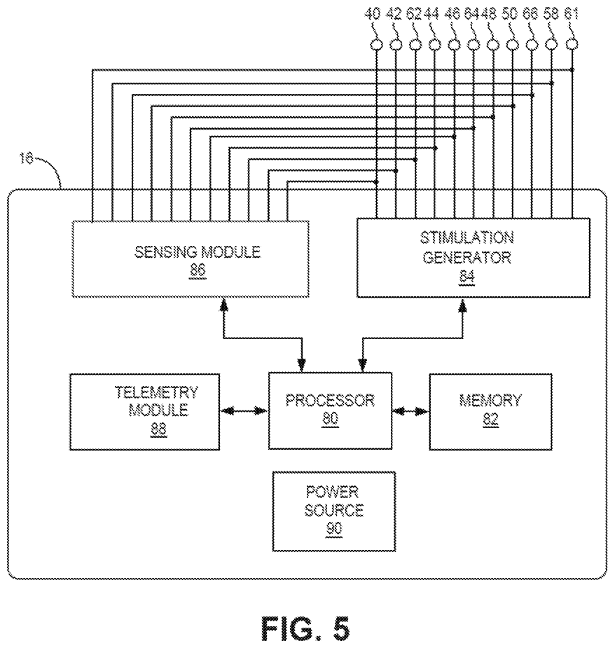

Electrodes 40, 42, 44, 46, 48 and 50 may sense electrical signals attendant to the depolarization and repolarization of heart 12. The electrical signals are conducted to IMD 16 via the respective leads 18, 20, 23. In some examples, IMD 16 also delivers pacing pulses via electrodes 40, 42, 44, 46, 48, 50 to cause depolarization of cardiac tissue of heart 12. In some examples, as illustrated in FIGS. 2B and 3B, IMD 16 includes one or more housing electrodes, such as housing electrode 58, which may be formed integrally with an outer surface of hermetically-sealed housing 60 of IMD 16 or otherwise coupled to housing 60. In some examples, housing electrode 58 may be defined by an uninsulated portion of an outward facing portion of housing 60 of IMD 16. Electrode 50 may be used for pacing and/or sensing of the cardiac conduction system tissue (e.g., His bundle or bundle branch tissue). Other divisions between insulated and uninsulated portions of housing 60 may be employed to define two or more housing electrodes. In some examples, housing electrode 58 includes substantially all of housing 60. Any of the electrodes 40, 42, 44, 46, 48 and 50 may be used for unipolar sensing or pacing in combination with housing electrode 58 or for bipolar sensing with two electrodes in the same pacing lead. In one or more embodiments, housing 60 may enclose a stimulation generator (see FIG. 5) that generates cardiac pacing pulses and defibrillation or cardioversion shocks, as well as a sensing module for monitoring the patient's heart rhythm.

Leads 18, 20, 23 may also include elongated electrodes 62, 64, 66, respectively, which may take the form of a coil. IMD 16 may deliver defibrillation shocks to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes.

Pressure sensor 38 may be coupled to one or more coiled conductors within lead 18. In FIG. 2B, pressure sensor 38 is located more distally on lead 18 than elongated electrode 62. In other examples, pressure sensor 38 may be positioned more proximally than elongated electrode 62, rather than distal to electrode 62. Further, pressure sensor 38 may be coupled to another one of the leads 20, 23 in other examples, or to a lead other than leads 18, 20, 23 carrying stimulation and sense electrodes. In addition, in some examples, pressure sensor 38 may be self-contained device that is implanted within heart 12, such as within the septum separating right ventricle 28 from left ventricle 32, or the septum separating right atrium 26 from left atrium 33. In such an example, pressure sensor 38 may wirelessly communicate with IMD 16.

FIG. 3B shows IMD 16 coupled to leads 18, 20, 22, 23. Right atrial (RA) lead 22 may extend through one or more veins and the vena cava, and into the right atrium 26 of heart 12. RA lead 22 may be connected to triple chamber IMD 16, e.g., using a Y-adaptor. IMD 16 may be used for cardiac rhythm therapy and defibrillation or cardioversion therapy (CRT-D). RA lead 22 may include electrodes that are the same or similar to the electrodes of lead 18, 20, 23, such as ring electrodes 40, 44 and 48, extendable helix tip electrodes 42, 46 and 50, and/or elongated electrodes 62, 64, 66, in the form of a coil.

FIGS. 3C-D show patient's heart 12 implanted with IMD 716 operably coupled to implantable medical electrical lead 723 to deliver bundle branch pacing according to one example of an IMD system 710. FIG. 3D is a close-up view of lead 723 in the patient's heart 12 of FIG. 3C. In some embodiments, electrical lead 723 may be the only lead implanted in the patient's heart 12. In other embodiments as discussed herein, there may be multiple leads implanted in the patient's heart 12. The one or more implantable electrodes may comprise a pacing electrode implantable proximate the cardiac conduction system, or may be implantable in the ventricular septum, to deliver cardiac conduction system pacing therapy, for examples.

In one embodiment, lead 723 may be configured for dual bundle branch pacing. Lead 723 may be the same as or similar to lead 23 (FIGS. 2A-B), except lead 723 is implanted near the bundle branches instead of, for example, the His bundle 13. As illustrated, lead 723 is implanted in the septal wall from RV 28 toward LV 32. Lead 723 may not pierce through the wall of LV 32 or extend into the LV chamber. Electrode 752 and tissue-piercing electrode assembly 761 may be disposed on a distal end portion of lead 723, which may also be described as a shaft. Electrode 752 and tissue-piercing electrode assembly 761 may be the same as or similar to electrode and tissue-piercing electrode assembly 50 (FIG. 2B), except electrode 752 is configured as a cathode electrode to sense or pace the RBB and electrode assembly 761 is configured to sense or pace the LBB, for example, during dual bundle branch pacing. Accordingly, electrode 752 may be implanted near RBB 8b, and electrode assembly 761 may be implanted near LBB 8a.

Electrode assembly 761 may be described as a unipolar cathode electrode, which may be implanted on the left side of the patient's septum. Electrode 752 may be described as a unipolar cathode electrode, which may be implanted on the right side of the patient's septum.

During dual bundle branch pacing, both electrode 752 and electrode assembly 761 (which also includes an electrode) may each deliver a cathodal pulse to achieve synchronized activation, or excitation, of RBB 8b and LBB 8a, which may result in synchronized activation of RV 28 and LV 32. In some embodiments, the pulses may be delivered at the same time to achieve synchrony. In other embodiments, the pulses may be delivered with a delay to achieve synchrony.

Lead 723 may include electrode 770 disposed more proximal to the electrode 752 and electrode assembly 761. Electrode 770 may be positioned in or near RA 26 and may function as an anode for cathodal pulses from electrode 752 and/or electrode assembly 761.

FIG. 3E is a conceptual diagram of lead 818 provided as a quadripolar lead carrying four electrodes 832, 834, 842 and 844 along a single lead body of lead 818. As described above, multiple pacing electrode configurations are selectable for delivering bundle branch pacing using electrodes 832, 834, 842 and/or 844 in various unipolar and/or bipolar pacing electrode vectors and selectable anode and cathode polarity assignments of each electrode 832, 834, 842 and 844. For example, tip electrode 832 and ring electrode 834 may deliver bipolar pacing pulses for capturing the LBB and ring electrodes 842 and 844 may deliver bipolar pacing pulses for capturing the RBB using two distinct bipolar pacing electrode vectors. Alternatively, a single bipolar pacing electrode vector, e.g., tip electrode 832 paired with any one of electrodes 834, 842 or 844, may be selected to deliver bipolar bilateral bundle branch pacing including cathodal and anodal capture. Any combination of two electrodes out of electrodes 832, 834, 842 and 844 may be selected in a bipolar pacing electrode vector with selectable anode and cathode polarities to achieve bipolar bilateral bundle branch pacing using a single bipolar pacing electrode vector. The selected electrode combination may be based on anodal and cathodal pacing capture thresholds of the LBB and RBB and/or the greatest improvement in ventricular electrical synchrony based on an analysis of ECG and/or EGM signals according to the techniques disclosed herein.

Furthermore, any of the electrodes 832, 834, 842 and 844 may be selected as a pacing cathode electrode in a unipolar pacing electrode vector including pacemaker housing 815 for pacing either the RBB or the LBB. Two unipolar pacing electrode vectors may be selected based on the lowest pacing pulse output required to capture both of the RBB and the LBB. However in some examples, correction of a ventricular conduction condition and improvement in ventricular electrical synchrony may be achieved by a cardiac conduction system pacing therapy that includes only single bundle branch pacing, e.g., only LBB pacing or only RBB pacing, using a selected bipolar or unipolar pacing electrode vector with only cathodal capture at the selected cathode electrode. As described herein, one or more processors, one or more processing circuits, and/or a computing apparatus of pacemaker 814 may analyze EGM signals to determine an advanced pacing delay for using in delivering cardiac conduction system pacing therapy during atrial fibrillation.

FIG. 3F is a conceptual diagram of pacemaker 814 configured as a multi-chamber pacemaker including an RA pacing and sensing lead 919 and a coronary sinus (CS) lead 992 in addition to lead 918 configured for delivering bundle branch pacing. While atrial lead 919 is not shown in FIG. 3E for the sake of clarity, it is to be understood that various pacing lead and electrode configurations described herein and shown in the accompanying drawings for delivering a cardiac conduction system pacing therapy may include an atrial lead 919 for providing atrial sensing and pacing and enabling pacemaker 814 to deliver atrial synchronized ventricular pacing. Furthermore, any of the leads carrying one or more electrodes shown in FIGS. 3E-F may be combined in other combinations than the illustrative examples presented herein, with more or fewer leads and/or electrodes than shown in these examples.

In FIG. 3F, pacemaker 814 is shown coupled to RA lead 919 carrying a pacing tip electrode 936 and proximal ring electrode 938 which may be used for sensing RA signals and delivering atrial pacing to the RA. The CS lead 992 may be advanced into the RA, through the coronary sinus ostium and into a cardiac vein of the LV for positioning electrodes 94a, 94b, 94c and 94d (collectively “CS electrodes 94”) epicardially along the LV myocardium for sensing EGM signals and pacing the LV myocardium. The CS lead 992 is shown as a quadripolar lead carrying four electrodes 94a-d that may be selected in various bipolar pacing electrode pairs for pacing the LV myocardial tissue and for sensing LV epicardial EGM signals. One of the CS electrodes 94 may be selected in combination with pacemaker housing 815 (or a coil electrode 935 if available) for delivering unipolar LV myocardial pacing and/or sensing unipolar ventricular EGM signals.

In this example, pacemaker 814 may be capable of delivering high voltage CV/DF shock therapies for cardioverting or defibrillating the heart in response to detecting a ventricular tachyarrhythmia. As such, lead 918 is shown carrying a coil electrode 35 for delivering high voltage shock pulses. One or more coil electrodes may be included along one or more of leads 918, 919 or 992 in various examples. A coil electrode such as coil electrode 935 may be selected in a unipolar pacing electrode vector with any of the lead-based tip or ring electrodes 932, 934, 936, 938 or 94 for sensing unipolar EGM signals for analysis and determination of ventricular conduction conditions. In some instances, coil electrode 935 may be used with housing 815 for sensing a far-field EGM signal for analyzing the QRS signal for determining a ventricular conduction condition and selecting a cardiac conduction system pacing therapy.

When pacing lead 918 is positioned for delivering bundle branch pacing, of one or both bundle branches, cardiac conduction system pacing therapy may be combined with ventricular myocardial pacing of the LV (using CS lead 992) to correct an LV conduction delay and achieve electrical and mechanical synchrony of the ventricles. As such, in some examples, one or more processors, one or more processing circuits, or a computing apparatus of the pacemaker 814 may select a cardiac conduction system pacing therapy plus LV myocardial pacing therapy that includes, for example, single or bilateral bundle branch pacing, e.g., using lead 918, combined with LV myocardial pacing using CS lead 992. As described below, one or more processors, one or more processing circuits, or a computing apparatus of the pacemaker 814 may receive EGM signals using one or more electrodes as described further herein (e.g., tip electrode to RV coil electrode unipolar EGM, tip electrode to housing electrode unipolar EGM, etc., as discussed below) to determine an advanced pacing delay as described further herein. In various examples, two or more EGM signals sensed using two or more respective sensing electrode vectors selected from the available electrodes coupled to pacemaker 814, and in some instances housing 815, may be analyzed by pacemaker 814 for determining an advanced pacing delay as described further herein.

The configuration of therapy system 10 illustrated in FIGS. 2A-4 are merely examples. In other examples, a therapy system may include epicardial leads and/or patch electrodes instead of or in addition to the transvenous leads 18, 20, 22 and/or cardiac conduction system pacing lead 23 illustrated in FIGS. 2A-4 or other configurations shown or described herein or incorporated by reference. Further, IMD 16 need not be implanted within patient 14. In examples in which IMD 16 is not implanted in patient 14, IMD 16 may deliver defibrillation shocks and other therapies to heart 12 via percutaneous leads that extend through the skin of patient 14 to a variety of positions within or outside of heart 12.

In other examples of therapy systems that provide electrical stimulation therapy to heart 12, such therapy systems may include any suitable number of leads coupled to IMD 16, and each of the leads may extend to any location within or proximate to heart 12. For example, therapy systems may include three transvenous leads located as illustrated in FIGS. 2A-4, and an additional lead located within or proximate to left atrium 33 (FIG. 1). As another example, therapy systems may include a single lead that extends from IMD 16 into right atrium 26 or right ventricle 28, or two leads that extend into a respective one of right ventricle 28 and right atrium 26. As another example, therapy systems may include a single lead that extends from IMD 16 into left ventricle 32 to deliver LVSP. An example of this type of therapy system is shown in FIGS. 3A and 3C-E. If four leads are required for therapy delivery, an IS-1 connector may be used in conjunction with Y-adaptor 25 extending from the RA port of the connector. The Y-adaptor allows two separate leads—e.g., right atrial lead and the bundle pacing bundle lead—to extend from the two separate legs of the “Y shape” while the single leg is inserted into connector block 34 on IMD 16.

FIG. 4 is a conceptual diagram illustrating another example of therapy system 70. Therapy system 70 shown in FIG. 4 may be useful for providing defibrillation and pacing pulses to heart 12. Therapy system 70 is similar to therapy system 10 of FIGS. 2A-B or 3A-B, but includes two leads 18, 23, rather than three leads. Therapy system 70 may utilize an IMD 16 configured to deliver, or perform, dual chamber pacing. Leads 18, 23 are implanted within right ventricle 28 and right atrium 26 to pace one or more portions of the cardiac conduction system such as the His bundle or one or both bundle branches, respectively.

Cardiac conduction system pacing lead 23 may be in the form of a helix (also referred to as a helical electrode) may be positioned proximate to, near, adjacent to, or in, area or portions of the cardiac conduction system such as, e.g., ventricular septum, triangle of Koch, the His bundle, left right bundle branch tissues, and/or right bundle branch tissue. Cardiac conduction system pacing lead 23 may be configured as a bipolar lead or as a quadripolar lead that may be used with a pacemaker device, a CRT-P device or a CRT-ICD.

FIG. 5 is a functional block diagram of one example configuration of IMD 16, which includes processor 80, memory 82, stimulation generator 84 (e.g., electrical pulse generator or signal generating circuit), sensing module 86 (e.g., sensing circuit), telemetry module 88, and power source 90. One or more components of IMD 16, such as processor 80, may be contained within a housing of IMD 16 (e.g., within a housing of a pacemaker). Telemetry module 88, sensing module 86, or both telemetry module 88 and sensing module 86 may be included in a communication interface. Memory 82 includes computer-readable instructions that, when executed by processor 80, cause IMD 16 and processor 80 to perform various functions attributed to IMD 16 and processor 80 herein. Memory 82 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Processor 80 may include any one or more of a microprocessor, a controller, a digital signal processor (DSP), an application specific integrated circuit (ASIC), a field-programmable gate array (FPGA), or equivalent discrete or integrated logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 herein may be embodied as software, firmware, hardware or any combination thereof. Processor 80 controls stimulation generator 84 to deliver stimulation therapy to heart 12 according to a selected one or more of therapy programs (e.g., optimization of the AV delay, VV delay, VV delay etc.), which may be stored in memory 82. Specifically, processor 80 may control stimulation generator 84 to deliver electrical pulses with amplitudes, pulse widths, frequency, or electrode polarities specified by the selected one or more therapy programs.

In some embodiments, RA lead 22 may be operably coupled to electrode 61, which may be used to monitor or pace the RA. Stimulation generator 84 may be electrically coupled to electrodes 40, 42, 44, 46, 48, 50, 58, 61, 62, 64, and 66, e.g., via conductors of respective lead 18, 20, 22, 23 or, in the case of housing electrode 58, via an electrical conductor disposed within housing 60 of IMD 16. Stimulation generator 84 may be configured to generate and deliver electrical stimulation therapy to heart 12. For example, stimulation generator 84 may deliver defibrillation shocks to heart 12 via at least two of electrodes 58, 62, 64, 66. Stimulation generator 84 may deliver pacing pulses via ring electrodes 40, 44, 48 coupled to leads 18, 20, 23, respectively, and/or helical electrodes 42, 46, and 50 of leads 18, 20, or 23, respectively. Cardiac conduction system pacing therapy can be delivered through cardiac conduction system lead 23 that is connected to an atrial, RV, or LV connection port of connector block 34. In some embodiments, the cardiac conduction system pacing therapy can be delivered through leads 18 and/or 23. In some examples, stimulation generator 84 delivers pacing, cardioversion, or defibrillation stimulation in the form of electrical pulses. In other examples, stimulation generator 84 may deliver one or more of these types of stimulation in the form of other signals, such as sine waves, square waves, or other substantially continuous time signals.

Stimulation generator 84 may include a switch module and processor 80 may use the switch module to select, e.g., via a data/address bus, which of the available electrodes are used to deliver defibrillation shocks or pacing pulses. The switch module may include a switch array, switch matrix, multiplexer, or any other type of switching device suitable to selectively couple stimulation energy to selected electrodes.

Sensing module 86 monitors signals from at least one of electrodes 40, 42, 44, 46, 48, 50, 58, 61, 62, 64 or 66 in order to monitor electrical activity of heart 12, e.g., via electrical signals, such as electrocardiogram (ECG) signals and/or electrograms (EGMs). Sensing module 86 may also include a switch module to select which of the available electrodes are used to sense the heart activity. In some examples, processor 80 may select the electrodes that function as sense electrodes via the switch module within sensing module 86, e.g., by providing signals via a data/address bus. In some examples, sensing module 86 includes one or more sensing channels, each of which may include an amplifier. In response to the signals from processor 80, the switch module may couple the outputs from the selected electrodes to one of the sensing channels.

In some examples, one channel of sensing module 86 may include an R-wave amplifier that receives signals from electrodes 44, 46, which are used for pacing and sensing proximate to left ventricle 32 of heart 12. Another channel may include another R-wave amplifier that receives signals from electrodes 40, 42, which are used for pacing and sensing in right ventricle 28 of heart 12. In some examples, the R-wave amplifiers may take the form of an automatic gain-controlled amplifier that provides an adjustable sensing threshold as a function of the measured R-wave amplitude of the heart rhythm.

In addition, in some examples, one channel of sensing module 86 may include a P-wave amplifier that receives signals from electrodes 48, 50, which are used for pacing and sensing in right atrium 26 of heart 12. In some examples, the P-wave amplifier may take the form of an automatic gain-controlled amplifier that provides an adjustable sensing threshold as a function of the measured P-wave amplitude of the heart rhythm. Examples of R-wave and P-wave amplifiers are described in U.S. Pat. No. 5,117,824 to Keimel et al., which issued on Jun. 2, 1992 and is entitled, “APPARATUS FOR MONITORING ELECTRICAL PHYSIOLOGIC SIGNALS”. Other amplifiers may also be used. Furthermore, in some examples, one or more of the sensing channels of sensing module 86 may be selectively coupled to housing electrode 58, or elongated electrodes 62, 64, or 66, with or instead of one or more of electrodes 40, 42, 44, 46, 48 or 50, e.g., for unipolar sensing of R-waves or P-waves in any of chambers 26, 28, or 32 of heart 12.

In some examples, sensing module 86 includes a channel that includes an amplifier with a relatively wider pass band than the R-wave or P-wave amplifiers or a high-resolution amplifier with relatively narrow-pass band for His bundle or bundle branch potential recording. Signals from the selected sensing electrodes that are selected for coupling to this wide-band amplifier may be provided to a multiplexer, and thereafter converted to multi-bit digital signals by an analog-to-digital converter for storage in memory 82 as an electrogram (EGM). In some examples, the storage of such EGMs in memory 82 may be under the control of a direct memory access circuit. Processor 80 may employ digital signal analysis techniques to characterize the digitized signals stored in memory 82 to detect and classify the patient's heart rhythm from the electrical signals. Processor 80 may detect and classify the heart rhythm of patient 14 by employing any of the numerous signal processing methodologies known in the art.

If IMD 16 is configured to generate and deliver pacing pulses to heart 12, processor 80 may include pacer timing and control module, which may be embodied as hardware, firmware, software, or any combination thereof. The pacer timing and control module may include a dedicated hardware circuit, such as an ASIC, separate from other processor 80 components, such as a microprocessor, or a software module executed by a component of processor 80, which may be a microprocessor or ASIC. The pacer timing and control module may include programmable counters which control the basic time intervals associated with DDD, VVI, DVI, VDD, AAI, DDI, DDDR, VVIR, DVIR, VDDR, AAIR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber in which an electrical signal is sensed, and the third letter may indicate the chamber in which the response to sensing is provided.

Intervals defined by the pacer timing and control module may include atrial and ventricular pacing escape intervals, refractory periods during which sensed P-waves and R-waves are ineffective to restart timing of the escape intervals, and the pulse widths of the pacing pulses. As another example, the pace timing and control module may define a blanking time period and provide signals from sensing module 86 to blank one or more channels, e.g., amplifiers, for a period during and after delivery of electrical stimulation to heart 12. The durations of these intervals may be determined by processor 80 in response to stored data in memory 82. The pacer timing and control module may also determine the amplitude of the cardiac pacing pulses. The pacer timing and control module may also determine an advanced pacing delay using the various pacing settings discussed above.

The advanced pacing delay is representative of a time period or a time period delay from delivery of a pacing pulse to a ventricular event as further described herein.

During pacing, escape interval counters within the pacer timing/control module may be reset upon sensing of R-waves and P-waves. Stimulation generator 84 may include pacer output circuits that are coupled, e.g., selectively by a switching module, to any combination of electrodes 40, 42, 44, 46, 48, 50, 58, 61, 62, or 66 appropriate for delivery of a bipolar or unipolar pacing pulse to one of the chambers of heart 12. Processor 80 may reset the escape interval counters upon the generation of pacing pulses by stimulation generator 84, and thereby control the basic timing of cardiac pacing functions, including anti-tachyarrhythmia pacing.

In some examples, processor 80 may operate as an interrupt driven device, and is responsive to interrupts from pacer timing and control module, where the interrupts may correspond to the occurrences of sensed P-waves and R-waves and the generation of cardiac pacing pulses. Any necessary mathematical calculations to be performed by processor 80 and any updating of the values or intervals controlled by the pacer timing and control module of processor 80 may take place following such interrupts. A portion of memory 82 may be configured as a plurality of recirculating buffers, capable of holding series of measured intervals, which may be analyzed by processor 80 in response to the occurrence of a pace or sense interrupt to determine whether the patient's heart 12 is presently exhibiting atrial or ventricular tachyarrhythmia.

Telemetry module 88 includes any suitable hardware, firmware, software or any combination thereof for communicating with another device, such as programmer 24 (FIG. 2A). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and the control signals for the telemetry circuit within telemetry module 88, e.g., via an address/data bus. In some examples, telemetry module 88 may provide received data to processor 80 via a multiplexer.

The various components of IMD 16 are coupled to power source 90, which may include a rechargeable or non-rechargeable battery. A non-rechargeable battery may be selected to last for several years, while a rechargeable battery may be inductively charged from an external device, e.g., on a daily or weekly basis.

The illustrative devices and methods described herein may provide and use an advanced pacing delay for using in cardiac therapy and cardiac resynchronization therapy (CRT) such as, e.g., cardiac conduction system pacing and left ventricular septal pacing (LVSP). The advanced pacing delay is representative of a time period for delivery of a pacing pulse (e.g., a cardiac conduction system pacing pulse such as a left bundle branch pacing pulse) to a ventricular event (e.g., breakout myocardial depolarization).

Using the advanced pacing delay, an IMD may effectively capture and pace the LV during atrial fibrillation. Illustrative CRT and determining effective capture of the left ventricle during CRT may be described in, for example, U.S. Pat. No. 9,339,656 B2 entitled “Methods and systems for identifying reasons for ineffective left ventricular capture in cardiac resynchronization therapy based on EGM morphology” filed on Feb. 28, 2014. The illustrative pacing therapy may be delivered using the advanced pacing delay to effectively capture the left ventricle (LV). The advanced pacing delay for cardiac conduction system pacing and the advanced pacing delay for LVSP may be the same or different values, as will be described further herein.

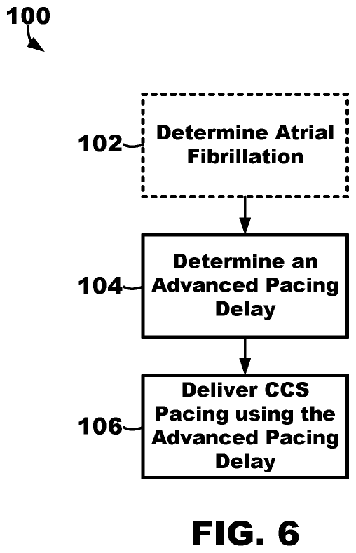

An illustrative method 100 of determining and using an advanced pacing delay for cardiac conduction system pacing during atrial fibrillation that may be utilized by the devices of FIGS. 2-5 is depicted in FIG. 6. The method 100 may optionally include (e.g., as signified by the dashed line box) determining atrial fibrillation 102. In some embodiments, the advanced pacing delay may be determined during an occurrence of atrial fibrillation, and in some other embodiments the advanced pacing delay may be determined when there is no atrial fibrillation occurring. It is to be understood that atrial fibrillation may be determined, or detected, using any one or more processes or techniques known in the art. Illustrative determination of atrial fibrillation may be described in, for example, U.S. Pat. No. 7,146,206 B2 entitled “Detection of cardiac arrhythmia using mathematical representation of standard ΔRR probability density histograms” filed on Mar. 6, 2003, and also in, for example, U.S. Pat. No. 7,233,822 B2 entitled “Combination of electrogram and intra-cardiac pressure to discriminate between fibrillation and tachycardia” filed on Jun. 29, 2004.

In response to determining that atrial fibrillation is occurring 102 or in the embodiment where process 102 is not utilized, the method 100 may further include determining an advanced pacing delay 104. When a cardiac conduction system pacing pulse is initiated, it may be described as being conducted or propagated very quickly through the cardiac conduction system, and then conducted or propagated more slowly through the ventricular myocardium before breakout myocardial depolarization occurs. The time it takes for the pacing pulse to travel from the cardiac conduction system pulse location (e.g., the left bundle branch, the right bundle branch, the His bundle, the AV node, the SA node, etc.) to breakout myocardial depolarization (e.g., the left ventricular or right ventricular myocardium) may be equated to the advanced pacing delay as used herein.

In some embodiments where the cardiac conduction system pulse is delivered to the left bundle branch, the advanced pacing delay is equal to the time it takes for the pulse to be conducted from the left bundle branch (LBB) to breakout LV myocardial depolarization. In such embodiments, the LBB also needs to be effectively captured such that a large portion of the breakout LV myocardial depolarization occurs via the activation of the LBB (as opposed to, for example, myocardial activation near an implanted electrode). Pacing according to, or using, the advanced pacing delay may effectively capture the LBB.

In response to or after determining the advanced pacing delay 104, the method 100 may further include delivering cardiac conduction system pacing using the advanced pacing delay 106. Various embodiments describing delivery of cardiac conduction system pacing are described above (e.g., using an IMD). Delivering cardiac conduction system pacing using the advanced pacing delay may be delivering cardiac conduction system pacing such that each pacing pulse is initiated “early” by a time equal to the advanced pacing delay. By early, it is meant that the pacing pulse is initiated prior to when it would otherwise be initiated. For example, during atrial fibrillation, the pacing pulses may be delivered such that they occur before or override intrinsic heart beats in order to effectively capture the LV. The advanced pacing delay may help define how much earlier each pacing pulse may be delivered in order to successfully override intrinsic heart rate, or intrinsic heart beats/intrinsic activation. This may provide pacing therapy (e.g., CRT) during atrial fibrillation that effectively captures the LV, which may result in improved stroke volume and cardiac output during atrial fibrillation.

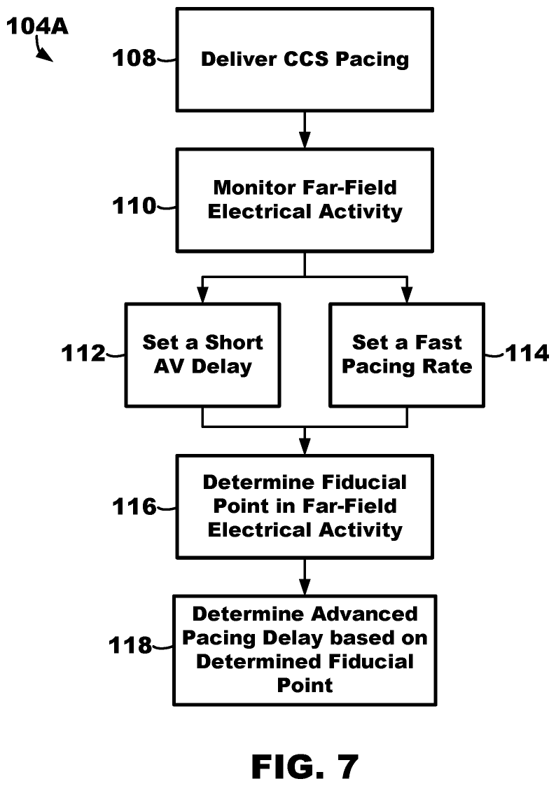

In one embodiment, delivering cardiac conduction system pacing using the advanced pacing delay 106 may comprise increasing a pacing rate of cardiac conduction system pacing based on the advanced pacing delay. Increasing the pacing rate of cardiac conduction system pacing may result in faster pacing of the patient's heart during atrial fibrillation in order to override intrinsic heart rate, and the advanced pacing delay may be used to ensure effective LBB capture and effective LV capture during the cardiac conduction system pacing. Various examples of determining an advanced pacing delay are described herein with respect to FIGS. 7-13.

An illustrative method of determining the advanced pacing delay 104A of the method of FIG. 6 for use in cardiac conduction system pacing is depicted in FIG. 7. As shown, the method 104A may include delivering test cardiac conduction system pacing to a patient's cardiac conduction system using a cardiac conduction system electrode implanted proximate a portion of the patient's cardiac conduction system 108. Delivering test cardiac conduction system pacing 108 occurs during atrial fibrillation, and may capture the cardiac conduction system (such capture of the cardiac conduction system as described above). The test cardiac conduction system pacing may be delivered by the systems and devices as described above with reference to FIGS. 2-5. The test cardiac conduction system pacing is referred to as “test” cardiac conduction system pacing because it is not being used for cardiac therapy in this circumstance, but is being used to determine an advanced pacing delay.

The method 104A may further include monitoring far-field electrical activity 110 as described above with reference to FIGS. 1-5. Monitoring electrical activity may include monitoring near- or far-field electrical signals using the systems and devices as described above. In one embodiment, monitoring electrical activity may include use of one or more surface electrodes (e.g., a standard 12-lead ECG), including electrodes positioned on the surface of a patient near or at the standard I, II, III, IV, V, and VI chest leads and/or upper right and left arm and lower right and left leg limb leads, for example. In the same embodiment, monitoring electrical activity may also include use of one or more implanted electrodes as discussed above with respect to FIGS. 1-5, including a tip electrode to an RV coil electrode unipolar EGM, a tip electrode to a housing electrode unipolar EGM, an RV coil electrode to a housing electrode unipolar EGM, and an atrial ring electrode to a housing electrode unipolar EGM, for example. A tip electrode may be an electrode located on the tip of any lead as described above. A coil electrode may be an electrode shaped in or on a coil and located proximal to the tip of the lead along the lead body. A ring electrode may be an electrode shaped in a ring around the lead body and located proximal to the tip of the lead along the lead body. A housing electrode may be an electrode located in or about the housing of the IMD, for example. Location identifiers such as RV are examples of possible electrode locations. The “atrial” location may be one of either atrium, for example. In alternative embodiments, bipolar EGM signals may be monitored, for example. In alternative embodiments, only surface electrodes or only implanted electrodes may be used. The one or more surface electrodes and the one or more implanted electrodes may include any other electrodes or electrode positions not specifically disclosed herein that are known to a person of ordinary skill in the art.

The method 104A may further include configuring, or setting, one or more parameters of the cardiac conduction system for use in determining the advanced pacing delay. For example, as shown, the method 104A may include setting a short AV delay 112 and/or setting a fast pacing rate 114. A short AV delay may between about 5 milliseconds (ms) and about 70 ms. In at least one embodiment, the short AV delay is 30 ms. In other embodiments, the short AV delay may be greater than or equal to 2 ms, greater than or equal to 15 ms, greater than or equal to 30 ms, greater than or equal to 40 ms, greater than or equal to 50 ms, greater than or equal to 60 ms, etc. and/or less than or equal to 80 ms, 75 ms, 70 ms, 60 ms, 50 ms, 40 ms, etc.

In another embodiment, the short AV delay may be determined as being related to, or as a function of, the patient's intrinsic AV delay. For example, the short AV delay may be configured as a selected or set percentage or ratio of the patient's intrinsic AV delay. In another embodiment, the short AV delay may be configured as a selected or set time period less than the patient's intrinsic AV delay.

A fast pacing rate may between about 10 beats per minute (bpm) faster and about 15 bpm faster than the patient's intrinsic or observed heart rate. In at least one embodiment, the fast pacing rate is 5 bpm faster. In other embodiments, the fast pacing rate may be greater than or equal to 2 bpm faster, greater than or equal to 12 bpm faster, greater than or equal to 18 bpm faster, greater than or equal to 20 bpm faster, greater than or equal to 22 bpm faster, greater than or equal to 25 bpm faster, etc. and/or less than or equal to 20 bpm, 15 bpm, 12 bpm, 10 bpm, 5 bpm, 2 bpm, etc.

In another embodiment, the fast pacing rate may be determined as being related to, or as a function of, the patient's intrinsic heart rate. For example, the fast pacing rate may be configured as a selected or set percentage or ratio of the patient's intrinsic heart rate. In another embodiment, the fast pacing rate may be configured as a set number of beats per minute faster than the patient's intrinsic heart rate.

The method 104A may further include monitoring far-field electrical activity using the one or more implantable electrodes during delivery of test cardiac conduction system pacing 110. Monitoring far-field electrical activity may be accomplished using sensing module 86, for example, as described above. Far-field electrical activity may include a far-field electrogram (EGM) signal as sensed from the at least one implanted electrode. In an alternative embodiment, far-field electrical activity may include a far-field electrocardiogram (ECG) signal as sensed from an external electrode, for example. The sensing module 86 or other sensing apparatus may monitor the far-field electrical activity (e.g., EGM signal) during the delivery of the test cardiac conduction system pacing 108.

The method 104A may further include determining a fiducial point within the far-field electrical activity 116. The fiducial point may be any measurable point within the far-field electrical activity signal. To determine the fiducial point, the devices and systems as described above may identify the fiducial point using any calculations as known to a person of ordinary skill in the art. Some examples of fiducial points may include one or more of: onset of a QRS morphology or any specific points therein, maximum signal amplitude, minimum signal amplitude, minimum and maximum signal slopes, etc. In one embodiment, the fiducial point is an earliest QRS deflection identified within the monitored far-field electrical activity following a test cardiac conduction system pacing pulse.

The method 104A may further include determining the advanced pacing delay based on the determined fiducial point 118. In one embodiment, the advanced pacing delay may be determined as the delay time period from delivery of a test cardiac conduction system pacing pulse of the test cardiac conduction system pacing to the determined fiducial point. Still further, for example, the advanced pacing delay may be determined as the delay time period from delivery of a test cardiac conduction system pacing pulse of the test cardiac conduction system pacing to the earliest QRS deflection monitored on a far-field EGM (i.e., the time from a LBB pacing pulse to breakout LV myocardial depolarization).

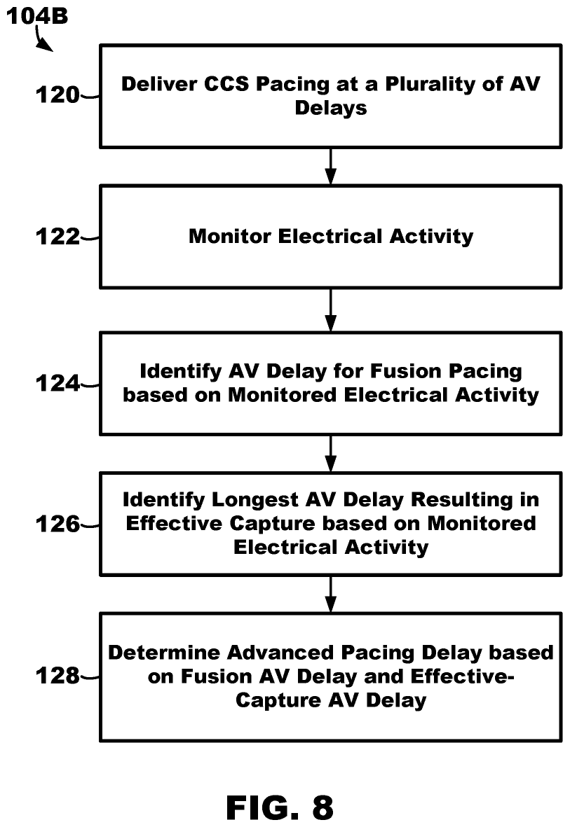

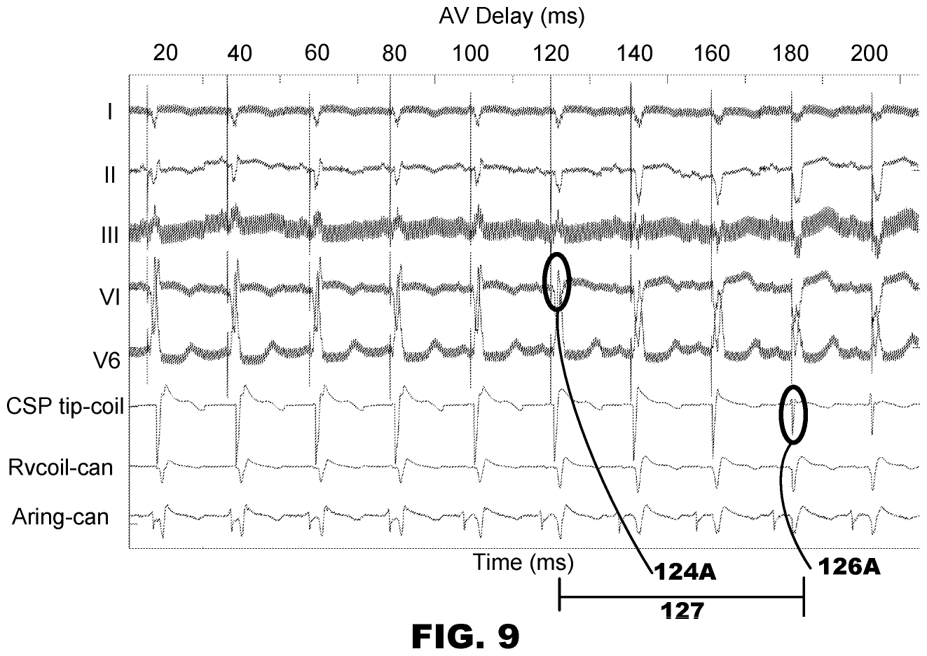

Another illustrative method of determining the advanced pacing delay 104B of the method of FIG. 6 for use in cardiac conduction system pacing is depicted in FIGS. 8 and 9. Exemplary electrocardiogram and electrogram signals monitored during delivery of cardiac conduction system pacing at a plurality of different AV delays are depicted in FIG. 9.

The method 104B (as illustrated in FIG. 8) may include delivering test cardiac conduction system pacing at a plurality of different AV delays over a testing time period 120, as shown in FIG. 9. Delivering test cardiac conduction system pacing as shown in method 104B is not usually done during atrial fibrillation, as the electrical activity during atrial fibrillation would likely be inconsistent and difficult to monitor. Each of the plurality of AV delays may correspond to a single paced heartbeat, for example. The plurality of AV delays may be any number of AV delays at AV delays ranging between about 20 ms and about 200 ms, as shown in FIG. 9, for example. In other embodiments, the AV delays may range between about 10 ms and about 300 ms, or between about 5 ms and about 250 ms, or between about 3 ms and about 400 ms, for example. The AV delays may be any AV delay within the range, and do not necessarily need to range in increments of 20 ms as shown in FIG. 9. For example, the AV delays may include every AV delay possible within the range, or may be stepped in increments of 1 ms or 5 ms or 10 ms or 25 ms, for example. The longest AV delay may be the AV delay which results in a pacing rate which does not override intrinsic heart beats, for example. In other words, the longest AV delay may be the AV delay which results in intrinsic heart beats and intrinsic activation, for example.

The method 104B may further include monitoring electrical activity using the one or more implantable electrodes during delivery of test cardiac conduction system pacing 122. The electrical activity may be monitored over any suitable time period, such as a time period including each of the plurality of different AV delays tested over consecutive heartbeats.

Monitoring electrical activity 122 may include monitoring near- or far-field electrical signals using the systems and devices as described above. In one embodiment, monitoring electrical activity may include use of one or more surface electrodes as discussed above. In another embodiment, monitoring electrical activity may include use of one or more implanted electrodes as discussed above. In yet another embodiment, monitoring electrical activity may include use of one or more surface electrodes and one or more implanted electrodes.

The method 104B may further include identifying a fusion AV delay of the plurality of AV delays based on the monitored electrical activity 124. The fusion AV delay is the AV delay where the test cardiac conduction system pacing results in fusion. Fusion is described above as synchronizing the timing of the LV pacing with the earliest activation on the RV chamber. In the illustrative embodiment depicted in FIG. 9, the fusion AV delay 124A occurs at the AV delay of 120 ms, for example. Identifying the fusion AV delay based on the monitored electrical activity may be completed, or performed, through analysis of the electrical activity such as analysis of the morphology, timing between fiducial points, etc., as known to a person of ordinary skill in the art. For example, the fusion AV delay may be the shortest AV delay where the electrical activity monitored from the VI surface electrode lead demonstrates loss of the R-wave morphology (as illustrated in FIG. 9). In another embodiment, the fusion AV delay may be the AV delay where the electrical activity monitored from the VI surface electrode lead demonstrates the narrowest QRS complex morphology. In another embodiment, the fusion AV delay may be the shortest AV delay which results in fusion. In another embodiment, the fusion AV delay may be the longest AV delay before fusion is first identified as described above.

The method 104B may further include identifying an effective-capture AV delay of the plurality of AV delays based on the monitored electrical activity 126. In the illustrative embodiment depicted in FIG. 9, the effective-capture AV delay 126A occurs at the AV delay of 180 ms, for example. Identifying the effective-capture AV delay based on the monitored electrical activity may be completed, or performed, through analysis of the electrical activity such as analysis of the morphology, timing between fiducial points, etc., as known to a person of ordinary skill in the art. For example, the effective-capture AV delay may be the longest AV delay where the test cardiac conduction system pacing results in effective capture of the patient's LV (or may be the shortest AV delay where the test cardiac conduction system pacing results in ineffective capture of the patient's LV as illustrated in FIG. 9). Effective capture of the LV may be determined using any devices, systems, or methods known to a person of ordinary skill in the art. For example, “EFFECTIVCRT™ DURING AF”′ feature as owned by Medtronic, Inc., classifies individual heartbeats as effective, ineffective, or sensed based on electrical activity morphology. The use of the trademark EFFECTIVCRT™ has been noted in this application.

In response to identifying the fusion AV delay of the plurality of AV delays 124 and the effective-capture AV delay of the plurality of AV delays 126, the method 104B may further include determining the advanced pacing delay based on the fusion AV delay and the effective-capture AV delay 128. For example, the advanced pacing delay may be determined by calculating the difference between the two identified AV delays. As illustrated in FIG. 9, the difference 127 between the AV delays and 124A and 126A is 60 ms. Thus, 60 ms is the advanced pacing delay determined using the monitored electrical activity of FIG. 9. In alternative embodiments, the advanced pacing delay may be a different calculated value, such as 50 ms, for example, and as described above.

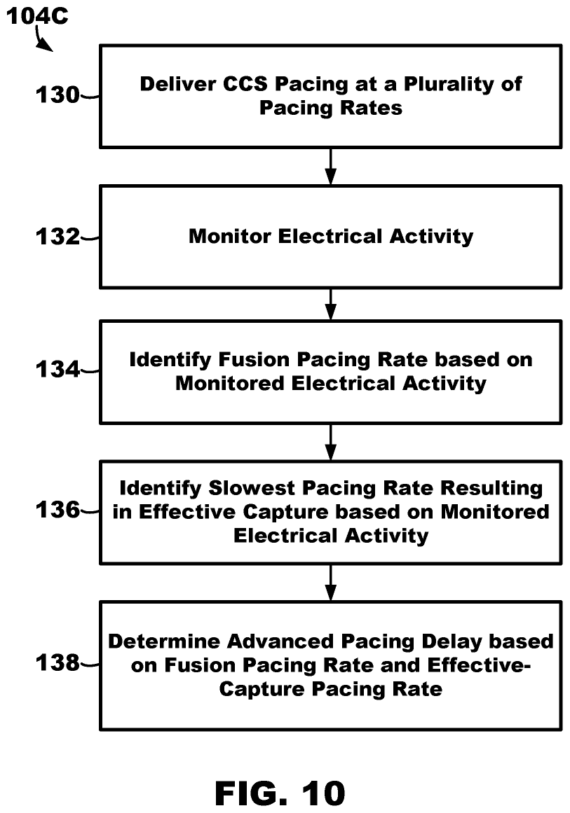

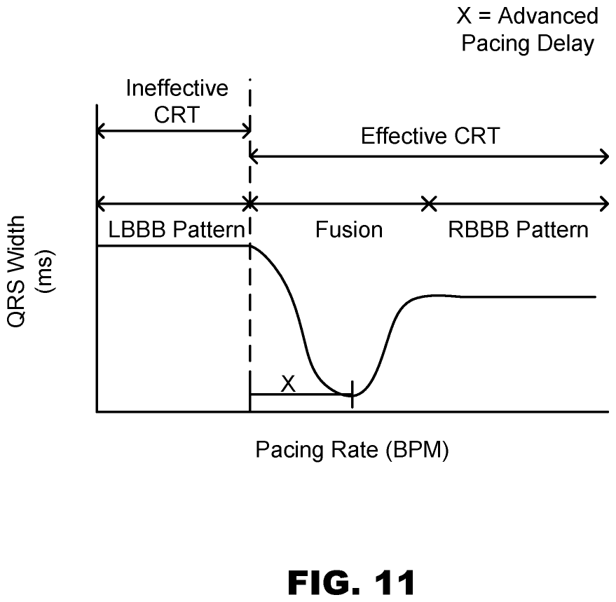

Another illustrative method of determining the advanced pacing delay 104C of the method of FIG. 6 for use in cardiac conduction system pacing is depicted in FIG. 10. An illustrative graph of QRS width as a function of pacing rate is depicted in FIG. 11 to illustrate the method 104C.

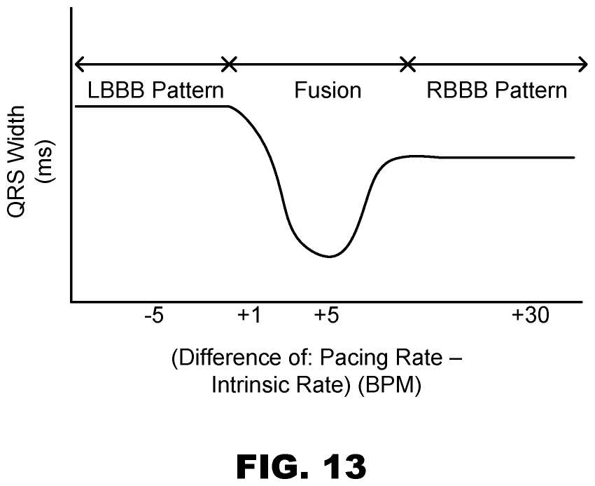

As illustrated in FIG. 11, in patients with the common conduction abnormality of left bundle branch block, for example, fast pacing during atrial fibrillation generally results in a wide QRS width which is a right bundle branch block (RBBB) pattern. Slow pacing during atrial fibrillation generally results in a wide QRS width which is a left bundle branch block (LBBB). Intrinsic activation (or pseudofusion) is similar to slow pacing, generally resulting in a wide QRS width, which is an LBBB pattern. Pseudofusion occurs when a ventricular pacing output is delivered, but it is too late to noticeably alter the intrinsic QRS morphology. Fusion, conversely, generally results in a narrower QRS width than either the LBBB or the RBBB patterns (as illustrated in FIGS. 11 and 13).

The method 104C may include delivering test cardiac conduction system pacing at a plurality of pacing rates over a testing time period 130, as shown in FIG. 10. Delivering test cardiac conduction system pacing as shown in method 104C is usually done during atrial fibrillation. Cardiac conduction pacing may be delivered at each of the plurality of delivered pacing rates over a single heartbeat, or multiple heartbeats, for example. The plurality of pacing rates may be any number of pacing rates at pacing rates ranging between about 15 bpm and about 20 bpm faster than the patient's intrinsic heart rate or the patient's most recently observed pacing rate. In other embodiments, the pacing rates may range between about 0 bpm and about 30 bpm faster than the intrinsic or observed rate, or between about 10 bpm and about 20 bpm faster than the intrinsic or observed rate, or between about 15 bpm and about 20 bpm faster than the intrinsic or observed rate, or between about 5 bpm and about 15 bpm faster than the intrinsic or observed rate, or between about 10 bpm and about 15 bpm faster than the intrinsic or observed rate, for example. The pacing rates may be any pacing rate within a selected range, as shown in FIG. 11.

The method 104C may further include monitoring electrical activity using the one or more implantable electrodes 132 during delivery of test cardiac conduction system pacing at the plurality of different pacing rates. The electrical activity may be monitored over any suitable time period, such as a time period including each of the plurality of pacing rates, each pacing rate tested over the span multiple heartbeats.

Monitoring electrical activity 132 may include monitoring near- or far-field electrical signals using the systems and devices as described above. In one embodiment, monitoring electrical activity may include use of one or more surface electrodes as discussed above. In another embodiment, monitoring electrical activity may include use of one or more implanted electrodes as discussed above. In yet another embodiment, monitoring electrical activity may include use of one or more surface electrodes and one or more implanted electrodes.

The method 104C may further include identifying a fusion pacing rate of the plurality of pacing rates based on the monitored electrical activity 134. The fusion pacing rate may be the pacing rate where the test cardiac conduction system pacing results in fusion. Identifying the fusion pacing rate based on the monitored electrical activity may be executed using identification or calculation of any morphology as known to a person of ordinary skill in the art. For example, the fusion pacing rate may occur at the pacing rate with the narrowest QRS width, as illustrated in FIG. 11.

The method 104C may further include identifying an effective-capture pacing rate of the plurality of pacing rates based on the monitored electrical activity 136. In the illustrative embodiment depicted in FIG. 11, the effective-capture pacing rate occurs at a slower pacing rate than the fusion pacing rate, for example. Identifying the effective-capture pacing rate based on the monitored electrical activity may be executed using identification or calculation of any morphology as known to a person of ordinary skill in the art. For example, the effective-capture pacing rate may be the slowest pacing rate where the test cardiac conduction system pacing results in effective capture of the patient's LV (or may be the fastest pacing rate where the test cardiac conduction system pacing results in ineffective capture of the patient's LV) as illustrated in FIG. 11. Effective capture of the LV may be determined using any devices, systems, or methods known to a person of ordinary skill in the art. For example, “EFFECTIVCRT™ DURING AF”′ feature as owned by Medtronic, Inc., classifies individual heartbeats as effective, ineffective, or sensed based on electrical activity morphology. The use of the trademark EFFECTIVCRT™ has been noted in this application.

The conduction disorder of LBBB is described in the above one or more embodiments. In alternate one or more embodiments, patients with RBBB will have a pattern which appears generally inverse to that of FIG. 11 (such that the pattern is flipped longitudinally in the x-direction and the x-axis values are inverted so that, for example, the x-axis provides an increasing advanced pacing delay from left to right, instead of an increasing pacing rate as currently illustrated in FIG. 11). In such alternate embodiments, if the advanced pacing delay is too short, an RBBB pattern results. If the advanced pacing delay is too long, an LBBB pattern results. Identifying a fusion pacing rate may be where the narrowest QRS width is identified, similar to the embodiments described herein. For patients with RBBB, selecting the AV delay that optimizes fusion between conduction system pacing and intrinsic activation may be similar to the one or more embodiments as described above.

In response to identifying the fusion pacing rate of the plurality of pacing rates 134 and the effective-capture pacing rate of the plurality of pacing rates 136, the method 104C may further include determining the advanced pacing delay based on the fusion pacing rate and the effective-capture pacing rate 138. For example, the advanced pacing delay may be determined by calculating the difference between the two identified pacing rates. As illustrated in FIG. 11, the pacing rates are calculated in bpm, while the advanced pacing delay, X, is calculated in ms. This conversion from a rate in bpm to a time delay in ms may be done in various ways. For example, the difference in the pacing rates may be converted from bpm to ms by multiplying both pacing rates by a conversion factor of minutes to milliseconds. One of ordinary skill in the art could understand various ways to convert a pacing rate in bpm to an advanced pacing delay in ms, or vice versa. The advanced pacing delay may be a value within ranges as described above.

Once the advanced pacing delay is determined using any of the above methods or embodiments, the advanced pacing delay may be used in delivering cardiac conduction system pacing (106 as illustrated in FIG. 6). In one embodiment, the advanced pacing delay may be determined once per patient, and then used as a consistent value thereafter. In one embodiment, the advanced pacing delay may be determined based on a population of patients, and then used as a default or nominal value for all subsequent patients. In one embodiment, the advanced pacing delay may be determined on a regular basis and further adapted over time after each subsequent determination. Each of the above embodiments may be used separately or together as desired.

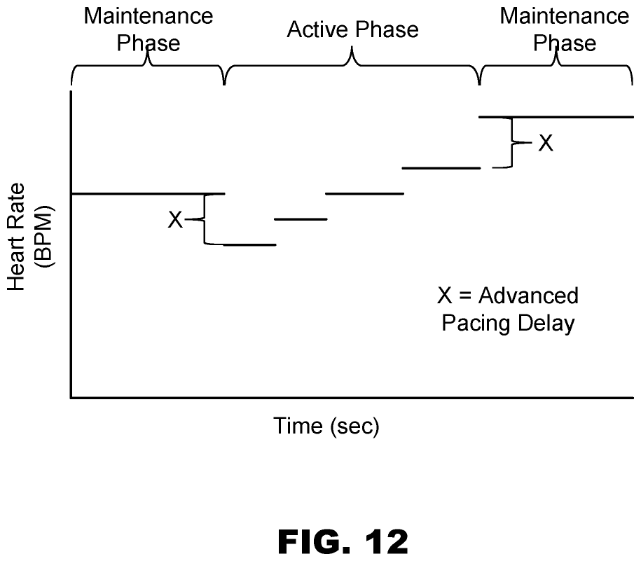

An exemplary graph of heart rate as a function of time during a maintenance phase, an active phase, and another maintenance phase is depicted in FIG. 12. In general, maintenance phase includes delivering cardiac conduction system pacing using the advanced pacing delay such as in, e.g., process 106 as illustrated in FIG. 6. In general, active phase includes removal of the advanced pacing delay from the pacing rate such that pacing cycle length is increased by X ms and a slower intrinsic or observed paced heart rate occurs. During active phase, the boundary between effective and ineffective capture of the LBB may be re-analyzed.

The method 100 as described above may further include delivering the cardiac conduction system pacing according to a maintenance pacing rate during a maintenance phase (as illustrated in the first maintenance phase of FIG. 12). During a maintenance phase, the cardiac conduction system pacing rate is increased to make sure that intrinsic heart rate during atrial fibrillation is overridden (as described above with respect to test cardiac conduction system pacing rates) and a significant portion of the LV myocardium is depolarized by the cardiac conduction system pacing pulse.

The method 100 may further include delivering the cardiac conduction system pacing according to a plurality of active pacing rates during an active phase (as illustrated in the active phase of FIG. 12). During transition from maintenance phase to active phase, the pacing cycle length is increased by a selected amount. The selected amount may be equivalent to the calculated advanced pacing delay, for example. Increasing the cycle length by the advanced pacing delay slows the paced heart rate. The slower paced heart rate is used to determine the transition point between ineffective and effective capture. The determination of transition point between ineffective and effective capture is used to ensure effective capture of the patient's cardiac conduction system. In other words, the pacing rate is decreased as pacing cycle length extends across more time. During active phase, pacing rate may be adjusted after each successive heartbeat. Active phase is not lengthy as the cardiac conduction system pacing will not be as effective with the extended pacing cycle length. Further, the power consumption during maintenance phase is lower than during active phase, so shorter active phases than maintenance phases preserve batter lifespan.

During active phase, if the pacing rate is determined to effectively capture the patient's cardiac conduction system (using the systems, devices, and methods discussed and incorporated herein), the pacing rate may be decreased (not illustrated in FIG. 12). If the pacing rate is determined to ineffectively capture the patient's cardiac conduction system, the pacing rate may be increased as illustrated in FIG. 12. In alternative embodiments, the cardiac conduction system pacing rate may be incrementally decreased during the active phase to find the boundary where ineffective LV capture occurs.

FIG. 13 is an exemplary graph of QRS width as a function of the difference between a pacing rate and an intrinsic rate. As shown, the pacing rate may be modified and the QRS width may be monitored during the active phase of FIG. 12 in order to re-analyze the boundary between effective and ineffective capture of the LBB and to provide continued effective capture of the LBB. The value of the difference between pacing rate and intrinsic rate may be as described above with respect to method 104C and FIG. 11.

In essence, the effective capture of the LBB may be continuously ensured based on QRS morphology, and more specifically, based on QRS width. As described above with respect to FIG. 11, fast pacing during atrial fibrillation generally results in a wide QRS width, which indicates a RBBB, and thus, may be referred to as a RBBB pattern. Intrinsic activation (or pseudofusion) generally results in a wide QRS width, which indicates a LBBB, and thus, may be referred to as a LBBB pattern. Pseudofusion occurs when a ventricular pacing output is delivered, but it is too late to noticeably alter the intrinsic QRS morphology. Fusion, conversely, generally results in a narrower QRS width than either the LBBB or the RBBB patterns (as illustrated in FIGS. 11 and 13).

For example, during active phase, pacing rate may be adjusted without the application of the advanced pacing delay while the QRS width may be monitored or measured. For instance, a plurality of difference pacing rates may be applied one at a time (e.g., a different rate for each beat, for a set of beats, etc.), for example, each without the application of the advanced pacing delay. Additionally, the plurality of different pacing rates may be defined in terms of the intrinsic rate. For example, as shown in FIG. 13, the different pacing rates extend from 5 bpm less (“−5”) than intrinsic to 30 bpm more (“+30”) than the intrinsic rate. More specifically, the plurality of different pacing rates may be any number of pacing rates ranging between about 0 bpm and about 20 bpm faster than the patient's intrinsic heart rate or the patient's most recently observed pacing rate. In other embodiments, the pacing rates may range between about 0 bpm and about 30 bpm faster than the intrinsic or observed rate, or between about 10 bpm and about 20 bpm faster than the intrinsic or observed rate, or between about 15 bpm and about 20 bpm faster than the intrinsic or observed rate, or between about 5 bpm and about 15 bpm faster than the intrinsic or observed rate, or between about 10 bpm and about 15 bpm faster than the intrinsic or observed rate, for example.

Adjustment of the pacing rate without the application of the advanced pacing delay allows for beat-to-beat adjustment of the pacing rate as described above with respect to FIG. 12. If there is effective capture of the LBB, pacing rate may be decreased. If there is ineffective capture of the LBB, pacing rate may be increased. QRS width may provide an indication of effective or ineffective capture of the LBB. For example, narrow QRS width signifying fusion may provide an indication of effective capture of the LBB. Further, for example, if the QRS width increases due to a change in paced rate, that would correspond to a movement away from effective capture, and toward ineffective capture of the LBB. If the QRS width decreases due to a change in paced rate, that would correspond to a movement toward effective capture of the LBB.

Thus, during the active phase, QRS width may be measured after each pacing pulse to determine whether effective capture of the LBB has occurred, and the pacing rate may be modified (e.g., increased or decreased) until the pacing rate that provides the narrowest QRS width is determined thereby signifying fusion, which may then be used to continuously ensure effective LBB capture. Moreover, it is to be understood that other variables besides QRS width signifying fusion may be used to ensure effective capture of the LBB in a similar fashion.

The conduction disorder of LBBB is described in the above one or more embodiments. In alternate one or more embodiments, patients with RBBB will have a pattern which appears generally inverse to that of FIG. 13 (such that the pattern is flipped longitudinally in the x-direction and the x-axis values are inverted so that, for example, the x-axis provides a decreasing pacing rate compared to intrinsic heart rate). Identifying a fusion pacing rate may be where the narrowest QRS width is identified, similar to the embodiments described herein.

In response to delivering the cardiac conduction system according to a plurality of active pacing rates during an active phase, the method 100 may further include determining the lowest active pacing rate that results in effective cardiac conduction system capture (using the systems, devices, and methods discussed and incorporated herein).

In response to determining the lowest active pacing rate that results in effective cardiac conduction system capture, the method 100 may further include setting the maintenance pacing rate based on the lowest active pacing rate and the previously selected pacing cycle increase length (as illustrated in the second maintenance phase of FIG. 12). During transition from active phase to maintenance phase, the pacing cycle length is decreased by the same selected amount as was used to increase the pacing cycle length when transitioning from the maintenance phase to the active phase. In other words, the pacing rate is increased as pacing cycle length decreases across less time. This may continue to ensure effective capture of the patient's cardiac conduction system while moving back to a maintenance phase.

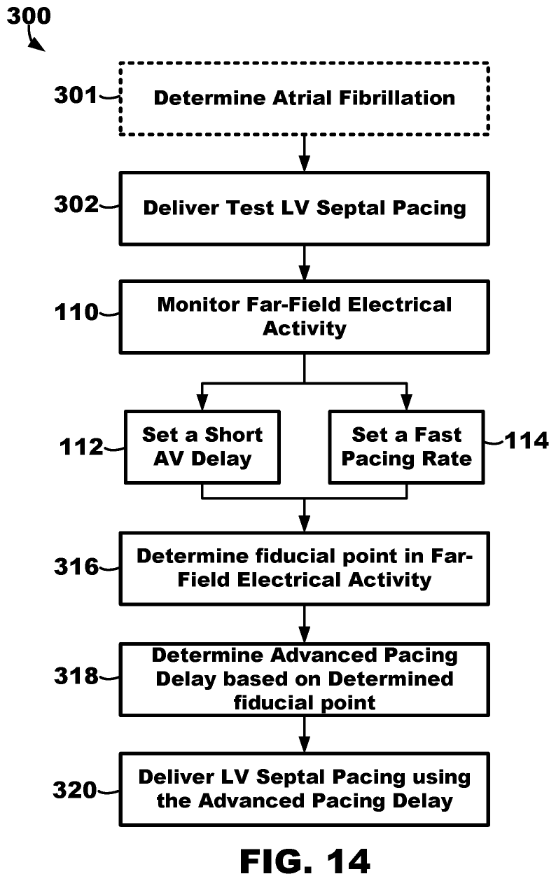

An illustrative method 300 of determining and using an advanced pacing delay for LV septal pacing (LVSP) during atrial fibrillation that may be utilized by the devices of FIGS. 2-5 is depicted in FIG. 14.

The method 300 may include determining an advanced pacing delay to capture a patient's LV, wherein the advanced pacing delay is representative of a time period from delivery of a pacing pulse (e.g., an LV septal pacing pulse) to a ventricular event (e.g., breakout myocardial depolarization). Method 300 may be very similar to method 100 and method 104A as described above. The method 300 may further include delivering therapy pacing to the patient's LV using one or more implanted electrodes comprising an electrode implanted proximate a portion of the patient's LV according to the advanced pacing delay.

The method 300 may optionally include determining atrial fibrillation 301, similar to the method 100 as described above. In some embodiments, the advanced pacing delay may be determined during an occurrence of atrial fibrillation, and in some other embodiments the advanced pacing delay may be determined when there is no atrial fibrillation occurring. In response to determining that atrial fibrillation is occurring or in the embodiment where process to determine atrial fibrillation is not utilized, the method 300 may further include determining the advanced pacing delay.

In response to determining the advanced pacing delay, the method 300 may further include delivering LV septal pacing using the advanced pacing delay. Various embodiments describing delivery of LV septal pacing are described above (e.g., using an IMD). Delivering LV septal pacing using the advanced pacing delay may be delivering LV septal pacing such that each pacing pulse is initiated “early” by a time equal to the advanced pacing delay. By early it is meant that the pacing pulse is initiated prior to when it would otherwise be initiated. For example, during atrial fibrillation, the pacing pulses may be delivered such that they occur before or override intrinsic heart beats in order to effectively capture the LV. The advanced pacing delay may help define how much earlier each pacing pulse may be delivered in order to successfully override intrinsic heart beats. This may provide pacing therapy (e.g., CRT) during atrial fibrillation that effectively captures the LV, which may result in improved stroke volume and cardiac output during atrial fibrillation, for example.

An illustrative method of determining the advanced pacing delay of the method 300 for use in LV septal pacing is depicted in FIG. 14. As shown, the method 300 may include delivering test LV septal pacing 302. Delivering test LV septal pacing 302 may occur during atrial fibrillation, and may capture the LV septal myocardium and other myocardial tissue within the LV. The LV septal pacing may be delivered by the systems and devices as described above with reference to FIGS. 2-5.

The method 300 may further include configuring, or setting, one or more parameters of the LV septal pacing for use in determining the advanced pacing delay. For example, as shown, the method 300 may include setting a short AV delay 112 and/or setting a fast pacing rate 114 as described above with respect to method 104A.

The method 300 may further include monitoring far-field electrical activity using at least one of the one or more implantable electrodes and one or more surface electrodes during delivery of test LV septal pacing 110 (as described above with respect to method 104A). The one or more implantable electrodes may be implanted in the LV or the ventricular septum, for example.

The method 300 may further include determining a fiducial point within the far-field electrical activity 316. The fiducial point may be any measurable point within the far-field electrical activity signal. To determine the fiducial point, the devices and systems as described above may identify the fiducial point using any calculations as known to a person of ordinary skill in the art. Some examples of fiducial points are described above. In one embodiment, the fiducial point is a maximum negative QRS deflection identified within the monitored far-field electrical activity following a test LV septal pacing pulse.

In response to determining the fiducial point within the far-field electrical activity 316, the method 300 may further include determining the advanced pacing delay based on the determined fiducial point 318. In one embodiment, the advanced pacing delay may be determined as the delay time period from delivery of a test LV septal pacing pulse of the test LV septal pacing to the determined fiducial point. The advanced pacing delay according to this embodiment may be as described above with respect to method 104A. Determining the advanced pacing delay based on the determined fiducial point 318 may be performed by the devices and systems as described above using any calculations as known to a person of ordinary skill in the art.

In response to determining the advanced pacing delay, the method 300 may further include delivering LV septal pacing using the advanced pacing delay 320. Various embodiments describing delivery of LV septal pacing are described above (e.g., using an IMD). Delivering LV septal pacing using the advanced pacing delay may be delivering LV septal pacing such that each pacing pulse is initiated “early” by a time equal to the advanced pacing delay. By early, it is meant that the pacing pulse is initiated prior to when it would otherwise be initiated. For example, during atrial fibrillation, the pacing pulses may be delivered such that they occur before or override intrinsic heart beats in order to effectively capture the LV. The advanced pacing delay may help define how much earlier each pacing pulse may be delivered in order to successfully override intrinsic heart rate, or intrinsic heart beats. This may provide pacing therapy (e.g., CRT) during atrial fibrillation that effectively captures the LV, which may result in improved stroke volume and cardiac output during atrial fibrillation.

Various examples have been described. These and other examples are within the scope of the following claims. For example, a single chamber, dual chamber, or triple chamber pacemakers (e.g., CRT-P) or ICDs (e.g., CRT-D) devices can be used to implement the illustrative methods described herein.

### ILLUSTRATIVE EXAMPLES

While the present disclosure is not so limited, an appreciation of various aspects of the disclosure will be gained through a discussion of the specific illustrative examples provided below. Various modifications of the illustrative examples, as well as additional examples of the disclosure, will become apparent herein.

Example Ex1: An implantable medical device comprising: one or more implantable electrodes to sense and pace a patient's heart, wherein the one or more implantable electrodes comprise a cardiac conduction system electrode positionable proximate a portion of the patient's cardiac conduction system; and a computing apparatus comprising processing circuitry, the computing apparatus operably coupled to the one or more implantable electrodes, wherein the computing apparatus is configured to:
determine an advanced pacing delay to capture the cardiac conduction system, wherein the advanced pacing delay is representative of a time period from delivery of a cardiac conduction system pacing pulse to a ventricular event; and
initiate delivery of cardiac conduction system pacing during atrial fibrillation using the cardiac conduction system electrode according to the advanced pacing delay.

Example Ex2: The implantable medical device as in Example Ex1, wherein initiating delivery of cardiac conduction system pacing during atrial fibrillation using the cardiac conduction system electrode according to the advanced pacing delay comprises increasing a pacing rate of the cardiac conduction system pacing based on the advanced pacing delay.

Example Ex3: The implantable medical device as any one of Examples Ex1-Ex2, wherein, to determine the advanced pacing delay, the computing apparatus is further configured to:
initiate delivery of test cardiac conduction system pacing during atrial fibrillation using the cardiac conduction system electrode according to one or more parameters to capture the cardiac conduction system, wherein the one or more parameters comprises a pacing rate faster than an intrinsic rate and an atrioventricular (AV) delay shorter than an intrinsic AV delay;
monitor far-field electrical activity using at least one of the one or more implantable electrodes and one or more surface electrodes during delivery of test cardiac conduction system pacing;
determine a fiducial point within the far-field electrical activity; and
determine the advanced pacing delay based on the fiducial point.

Example Ex4: The implantable medical device as in Example Ex3, wherein the advanced pacing delay is a delay time period from delivery of a test cardiac conduction system pacing pulse of the test cardiac conduction system pacing to the fiducial point.

Example Ex5: The implantable medical device as in any one of Examples Ex3-Ex4, wherein the fiducial point is an earliest QRS deflection identified within the monitored far-field electrical activity following the test cardiac conduction system pacing pulse.

Example Ex6: The implantable medical device as in any one of Examples Ex1-Ex5, wherein, to determine the advanced pacing delay, the computing apparatus is further configured to:
initiate delivery of test cardiac conduction system pacing using the cardiac conduction system electrode at a plurality of AV delays over a testing time period;
monitor electrical activity using at least one of the one or more implantable electrodes and one or more surface electrodes during delivery of test cardiac conduction system pacing at the plurality of AV delays over the testing time period;
identify a fusion AV delay of the plurality of AV delays based on the monitored electrical activity, wherein the fusion AV delay is the shortest AV delay where the test cardiac conduction system pacing results in fusion;
identify an effective-capture AV delay of the plurality of AV delays based on the monitored electrical activity, wherein the effective-capture AV delay is the longest AV delay where the test cardiac conduction system pacing effectively captures the patient's cardiac conduction system; and
determine the advanced pacing delay based on the fusion AV delay and the effective-capture AV delay.

Example Ex7: The implantable medical device as in any one of Examples Ex1-Ex6, wherein to determine the advanced pacing delay, the computing apparatus is further configured to:
determine that the patient is undergoing atrial fibrillation;
initiate delivery of test cardiac conduction system pacing at a plurality of pacing rates over a testing time period using the cardiac conduction system electrode in response to the determination that the patient is undergoing atrial fibrillation;
monitor electrical activity using at least one of the one or more implantable electrodes or one or more surface electrodes during delivery of test cardiac conduction system pacing at the plurality of pacing rates over the testing time period;
identify a fusion pacing rate of the plurality of pacing rates based on the monitored electrical activity, wherein the fusion pacing rate is the pacing rate where the test cardiac conduction system pacing results in fusion;
identify an effective-capture pacing rate of the plurality of pacing rates based on the monitored electrical activity, wherein the effective-capture pacing rate is the slowest pacing rate where the test cardiac conduction system pacing effectively captures the patient's cardiac conduction system; and
determine the advanced pacing delay based on the fusion pacing rate and the effective-capture pacing rate.

Example Ex8: The implantable medical device as in Example Ex7, wherein the advanced pacing delay is the difference between a fusion paced RR interval (e.g., the interval between two successive R-waves) provided by the fusion pacing rate and an effective-capture paced RR interval provided the effective-capture pacing rate.

Example Ex. 9: The implantable medical device as in any one of Examples Ex1-Ex8, wherein the cardiac conduction system electrode is implantable in the ventricular septum to deliver cardiac conduction system pacing therapy.

Example Ex10: An implantable medical device comprising:
one or more implantable electrodes to sense and pace a patient's heart, wherein the one or more implantable electrodes comprise an electrode positionable proximate a left ventricle of the patient's heart; and
a computing apparatus comprising processing circuitry, the computing apparatus operably coupled to the one or more implantable electrodes, wherein the computing apparatus is configured to:
determine an advanced pacing delay to capture the ventricular myocardium, wherein the advanced pacing delay is representative of a time period from delivery of a LV septal pacing pulse to a ventricular event; and
initiate delivery of a LV septal pacing during atrial fibrillation using the electrode according to the advanced pacing delay,
wherein to determine the advanced pacing delay, the computing apparatus is further configured to:
   initiate delivery of test LV septal pacing during atrial fibrillation using the electrode according to one or more parameters to capture the ventricular myocardium, wherein the one or more parameters comprises an atrioventricular (AV) delay shorter than an intrinsic AV delay;
   monitor a far-field electrogram using at least one of the one or more implantable electrodes and one or more surface electrodes during the test LV septal pacing;
   determine a fiducial point within the far-field electrogram; and
   determine the advanced pacing delay based on the fiducial point,
wherein the advanced pacing delay is a delay time period from delivery of a test LV septal pacing pulse of the test LV septal pacing to the fiducial point, and
wherein the fiducial point is a maximum negative QRS deflection identified within the far-field electrogram.

Example Ex11: A method comprising:
determining an advanced pacing delay to capture a patient's cardiac conduction system, wherein the advanced pacing delay is representative of a time period from delivery of a cardiac conduction system pacing pulse to a ventricular event; and
delivering cardiac conduction system pacing during atrial fibrillation using a cardiac conduction system electrode positioned proximate a portion of a patient's cardiac conduction system according to the advanced pacing delay.

Example Ex12: The method as in Example Ex11, wherein delivering cardiac conduction system pacing during atrial fibrillation using the cardiac conduction system electrode positioned proximate the portion of the patient's cardiac conduction system according to the advanced pacing delay comprises increasing a pacing rate of the cardiac conduction system pacing based on the advanced pacing delay.

Example Ex13: The method as in any one of Examples Ex11-Ex12, wherein determining the advanced pacing delay further comprises:
delivering test cardiac conduction system pacing during atrial fibrillation using the cardiac conduction system electrode positioned proximate the portion of the patient's cardiac conduction system according to one or more parameters to capture the cardiac conduction system, wherein the one or more parameters comprises a pacing rate faster than an intrinsic rate and an atrioventricular (AV) delay shorter than an intrinsic AV delay;
monitoring far-field electrical activity using at least one of one or more implantable electrodes and one or more surface electrodes during delivery of test cardiac conduction system pacing;
determining a fiducial point within the far-field electrical activity; and
determining the advanced pacing delay based on the fiducial point.

Example Ex14: The method as in Example Ex13, wherein the advanced pacing delay is a delay time period from delivery of a test cardiac conduction system pacing pulse of the test cardiac conduction system pacing to the fiducial point.

Example Ex15: The method as in any one of Examples Ex 13-Ex14, wherein the fiducial point is an earliest QRS deflection identified within the monitored far-field electrical activity following the test cardiac conduction system pacing pulse.

Example Ex 16: The method as in any one of Examples Ex11-Ex15, wherein determining the advanced pacing delay further comprises:
delivering test cardiac conduction system pacing using the cardiac conduction system electrode at a plurality of AV delays over a testing time period;
monitoring electrical activity using at least one of one or more implantable electrodes and one or more surface electrodes during delivery of test cardiac conduction system pacing at the plurality of AV delays over the testing time period;
identifying a fusion AV delay of the plurality of AV delays based on the monitored electrical activity, wherein the fusion AV delay is the shortest AV delay where the test cardiac conduction system pacing results in fusion;
identifying an effective-capture AV delay of the plurality of AV delays based on the monitored electrical activity, wherein the effective-capture AV delay is the longest AV delay where the test cardiac conduction system pacing effectively captures the patient's cardiac conduction system; and
determining the advanced pacing delay based on the fusion AV delay and the effective-capture AV delay.

Example Ex17: The method as in any one of Examples Ex11-Ex16, wherein determining the advanced pacing delay further comprises:
determining that the patient is undergoing atrial fibrillation;
delivering test cardiac conduction system pacing at a plurality of pacing rates over a testing time period using the cardiac conduction system electrode in response to the determination that the patient is undergoing atrial fibrillation;
monitoring electrical activity using at least one of one or more implantable electrodes or one or more surface electrodes during delivery of test cardiac conduction system pacing at the plurality of pacing rates over the testing time period;
identifying a fusion pacing rate of the plurality of pacing rates based on the monitored electrical activity, wherein the fusion pacing rate is the pacing rate where the test cardiac conduction system pacing results in fusion;
identifying an effective-capture pacing rate of the plurality of pacing rates based on the monitored electrical activity, wherein the effective-capture pacing rate is the slowest pacing rate where the test cardiac conduction system pacing effectively captures the patient's cardiac conduction system; and
determining the advanced pacing delay based on the fusion pacing rate and the effective-capture pacing rate.

Example Ex18: The implantable medical device as in Example Ex17, wherein the advanced pacing delay is the difference between a fusion paced RR interval provided by the fusion pacing rate and an effective-capture paced RR interval provided the effective-capture pacing rate.

Example Ex 19: The method as in any one of Examples Ex 11-Ex18, wherein the cardiac conduction system electrode is implantable in the ventricular septum to deliver cardiac conduction system pacing therapy.

Example Ex20: A method comprising:
determining an advanced pacing delay to capture a patient's left ventricle, wherein the advanced pacing delay is representative of a time period from delivery of a pacing pulse to a ventricular event; and
delivering LV septal pacing during atrial fibrillation using an electrode positioned proximate a left ventricle of the patient's heart according to the advanced pacing delay,
wherein determining the advanced pacing delay further comprises:
   delivering test LV septal pacing using the electrode during atrial fibrillation according to one or more parameters to capture the ventricular myocardium, wherein the one or more parameters comprises an atrioventricular (AV) delay shorter than an intrinsic AV delay;
   monitoring a far-field electrogram using at least one of one or more implantable electrodes and one or more surface electrodes during the test LV septal pacing;
   determining a fiducial point within the far-field electrogram; and
   determining the advanced pacing delay based on the fiducial point,
wherein the advanced pacing delay is a delay time period from delivery of a test LV septal pacing pulse of the test LV septal pacing to the fiducial point, and
wherein the fiducial point is a maximum negative QRS deflection identified within the far-field electrogram.

This disclosure has been provided with reference to illustrative embodiments and examples and is not meant to be construed in a limiting sense. As described previously, one skilled in the art will recognize that other various illustrative applications may use the techniques as described herein to take advantage of the beneficial characteristics of the devices and methods described herein. Various modifications of the illustrative embodiments and examples will be apparent upon reference to this description.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

All references and publications cited herein are expressly incorporated herein by reference in their entirety for all purposes, except to the extent any aspect directly contradicts this disclosure.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The recitation of numerical ranges by endpoints includes all numbers subsumed within that range (e.g. 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality (for example, a mobile user device may be operatively coupled to a cellular network transmit data to or receive data therefrom).

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” It will be understood that “consisting essentially of,” “consisting of,” and the like are subsumed in “comprising,” and the like.

The term “and/or” means one or all of the listed elements or a combination of at least two of the listed elements.

The phrases “at least one of,” “comprises at least one of,” and “one or more of”′ followed by a list refers to any one of the items in the list and any combination of two or more items in the list.

1. An implantable medical device comprising:
a computing apparatus comprising processing circuitry, the computing apparatus operably coupled to one or more implantable electrodes to sense and pace a patient's heart, wherein the computing apparatus is configured to:
determine an advanced pacing delay to capture the patient's cardiac conduction system, wherein the advanced pacing delay is representative of a time period from delivery of a cardiac conduction system pacing pulse to a ventricular event; and
initiate delivery of cardiac conduction system pacing during atrial fibrillation using a cardiac conduction system electrode of the one or more implantable electrodes according to the advanced pacing delay, the cardiac conduction system electrode positioned proximate a portion of the patient's cardiac conduction system.

2. The implantable medical device of claim 1, wherein initiating delivery of cardiac conduction system pacing during atrial fibrillation using the cardiac conduction system electrode according to the advanced pacing delay comprises increasing a pacing rate of the cardiac conduction system pacing based on the advanced pacing delay.

3. The implantable medical device of claim 1, wherein, to determine the advanced pacing delay, the computing apparatus is further configured to:
initiate delivery of test cardiac conduction system pacing during atrial fibrillation using the patient's cardiac conduction system electrode according to one or more parameters to capture the cardiac conduction system, wherein the one or more parameters comprises a pacing rate faster than an intrinsic rate and an atrioventricular (AV) delay shorter than an intrinsic AV delay;
monitor far-field electrical activity using at least one of the one or more implantable electrodes and one or more surface electrodes during delivery of test cardiac conduction system pacing;
determine a fiducial point within the far-field electrical activity; and
determine the advanced pacing delay based on the fiducial point.

4. The implantable medical device of claim 3, wherein the advanced pacing delay is a delay time period from delivery of a test cardiac conduction system pacing pulse of the test cardiac conduction system pacing to the fiducial point.

5. The implantable medical device of claim 3, wherein the fiducial point is an earliest QRS deflection identified within the monitored far-field electrical activity following the test cardiac conduction system pacing.

6. The implantable medical device of claim 1, wherein, to determine the advanced pacing delay, the computing apparatus is further configured to:
initiate delivery of test cardiac conduction system pacing using the cardiac conduction system electrode at a plurality of AV delays over a testing time period;
monitor electrical activity using at least one of the one or more implantable electrodes and one or more surface electrodes during delivery of test cardiac conduction system pacing at the plurality of AV delays over the testing time period;
identify a fusion AV delay of the plurality of AV delays based on the monitored electrical activity, wherein the fusion AV delay is the shortest AV delay where the test cardiac conduction system pacing results in fusion;
identify an effective-capture AV delay of the plurality of AV delays based on the monitored electrical activity, wherein the effective-capture AV delay is the longest AV delay where the test cardiac conduction system pacing effectively captures the patient's cardiac

7. The implantable medical device of claim 1, wherein to determine the advanced pacing delay, the computing apparatus is further configured to:
determine that the patient is undergoing atrial fibrillation;
initiate delivery of test cardiac conduction system pacing at a plurality of pacing rates over a testing time period using the cardiac conduction system electrode in response to the determination that the patient is undergoing atrial fibrillation;
monitor electrical activity using at least one of the one or more implantable electrodes or one or more surface electrodes during delivery of test cardiac conduction system pacing at the plurality of pacing rates over the testing time period;
identify a fusion pacing rate of the plurality of pacing rates based on the monitored electrical activity, wherein the fusion pacing rate is the pacing rate where the test cardiac conduction system pacing results in fusion;
identify an effective-capture pacing rate of the plurality of pacing rates based on the monitored electrical activity, wherein the effective-capture pacing rate is the slowest pacing rate where the test cardiac conduction system pacing effectively captures the patient's cardiac conduction system; and
determine the advanced pacing delay based on the fusion pacing rate and the effective-capture pacing rate.

8. The implantable medical device of claim 7, wherein the advanced pacing delay is the difference between a fusion paced RR interval provided by the fusion pacing rate and an effective-capture paced RR interval provided the effective-capture pacing rate.

9-15. (canceled)

16. The implantable medical device of claim 1, wherein the cardiac conduction system electrode is implanted in the patient's ventricular septum.

17. An implantable medical device comprising:
a computing apparatus comprising processing circuitry, the computing apparatus operably coupled to one or more implantable electrodes to sense and pace a patient's heart, wherein the computing apparatus is configured to:
determine an advanced pacing delay to capture the ventricular myocardium, wherein the advanced pacing delay is representative of a time period from delivery of a LV septal pacing pulse to a ventricular event; and
initiate delivery of a LV septal pacing during atrial fibrillation using a septal electrode of the one or more implantable electrodes positionable proximate the left ventricular septum of the patient's heart according to the advanced pacing delay,
wherein to determine the advanced pacing delay, the computing apparatus is further configured to:
initiate delivery of test LV septal pacing during atrial fibrillation using the septal electrode according to one or more parameters to capture the ventricular myocardium, wherein the one or more parameters comprises an atrioventricular (AV) delay shorter than an intrinsic AV delay;
monitor a far-field electrogram using at least one of the one or more implantable electrodes and one or more surface electrodes during the test LV septal pacing;
determine a fiducial point within the far-field electrogram; and
determine the advanced pacing delay based on the fiducial point,
wherein the advanced pacing delay is a delay time period from delivery of a test LV septal pacing pulse of the test LV septal pacing to the fiducial point, and
wherein the fiducial point is a maximum negative QRS deflection identified within the far-field electrogram.

18. A method via processing circuitry of an implantable medical device comprising:
determining an advanced pacing delay to capture a patient's cardiac conduction system, wherein the advanced pacing delay is representative of a time period from delivery of a cardiac conduction system pacing pulse to a ventricular event; and
initiating delivery of cardiac conduction system pacing during atrial fibrillation using a cardiac conduction system electrode positioned proximate a portion of a patient's cardiac conduction system according to the advanced pacing delay.

19. The method of claim 18, wherein initiating delivery of cardiac conduction system pacing during atrial fibrillation using the cardiac conduction system electrode positioned proximate the portion of the patient's cardiac conduction system according to the advanced pacing delay comprises increasing a pacing rate of the cardiac conduction system pacing based on the advanced pacing delay.

20. The method of claim 18, wherein determining the advanced pacing delay further comprises:
initiating delivery of test cardiac conduction system pacing during atrial fibrillation using the cardiac conduction system electrode positioned proximate the portion of the patient's cardiac conduction system according to one or more parameters to capture the cardiac conduction system, wherein the one or more parameters comprises a pacing rate faster than an intrinsic rate and an atrioventricular (AV) delay shorter than an intrinsic AV delay;
monitoring far-field electrical activity using at least one of one or more implantable electrodes and one or more surface electrodes during delivery of test cardiac conduction system pacing;
determining a fiducial point within the far-field electrical activity; and
determining the advanced pacing delay based on the fiducial point.

21. The method of claim 20, wherein the advanced pacing delay is a delay time period from delivery of a test cardiac conduction system pacing pulse of the test cardiac conduction system pacing to the fiducial point.

22. The method of claim 20, wherein the fiducial point is an earliest QRS deflection identified within the monitored far-field electrical activity following the test cardiac conduction system pacing.

23. The method of claim 18, wherein determining the advanced pacing delay further comprises:
initiating delivery of test cardiac conduction system pacing using the cardiac conduction system electrode at a plurality of AV delays over a testing time period;
monitoring electrical activity using at least one of one or more implantable electrodes and one or more surface electrodes during delivery of test cardiac conduction system pacing at the plurality of AV delays over the testing time period;
identifying a fusion AV delay of the plurality of AV delays based on the monitored electrical activity, wherein the fusion AV delay is the shortest AV delay where the test cardiac conduction system pacing results in fusion;
identifying an effective-capture AV delay of the plurality of AV delays based on the monitored electrical activity, wherein the effective-capture AV delay is the longest AV delay where the test cardiac conduction system pacing effectively captures the patient's cardiac conduction system; and
determining the advanced pacing delay based on the fusion AV delay and the effective-capture AV delay.

24. The method of claim 18, wherein determining the advanced pacing delay further comprises:
determining that the patient is undergoing atrial fibrillation;
initiating delivery of test cardiac conduction system pacing at a plurality of pacing rates over a testing time period using the cardiac conduction system electrode in response to the determination that the patient is undergoing atrial fibrillation;
monitoring electrical activity using at least one of one or more implantable electrodes or one or more surface electrodes during delivery of test cardiac conduction system pacing at the plurality of pacing rates over the testing time period;
identifying a fusion pacing rate of the plurality of pacing rates based on the monitored electrical activity, wherein the fusion pacing rate is the pacing rate where the test cardiac conduction system pacing results in fusion;
identifying an effective-capture pacing rate of the plurality of pacing rates based on the monitored electrical activity, wherein the effective-capture pacing rate is the slowest pacing rate where the test cardiac conduction system pacing effectively captures the patient's cardiac conduction system; and
determining the advanced pacing delay based on the fusion pacing rate and the effective-capture pacing rate.

25. The method of claim 24, wherein the advanced pacing delay is the difference between a fusion paced RR interval provided by the fusion pacing rate and an effective-capture paced RR interval provided the effective-capture pacing rate.

26. The method of claim 18, wherein the cardiac conduction system electrode is implanted in the patient's ventricular septum to deliver cardiac conduction system pacing therapy.

27. A method via processing circuitry of an implantable medical device comprising:
determining an advanced pacing delay to capture a patient's left ventricle, wherein the advanced pacing delay is representative of a time period from delivery of a pacing pulse to a ventricular event; and
initiating delivery of left ventricular (LV) septal pacing during atrial fibrillation using an electrode positioned proximate a left ventricle of the patient's heart according to the advanced pacing delay,
wherein determining the advanced pacing delay further comprises:
delivering test LV septal pacing using the electrode during atrial fibrillation according to one or more parameters to capture the ventricular myocardium, wherein the one or more parameters comprises an atrioventricular (AV) delay shorter than an intrinsic AV delay;
monitoring a far-field electrogram using at least one of one or more implantable electrodes and one or more surface electrodes during the test LV septal pacing;
determining a fiducial point within the far-field electrogram; and
determining the advanced pacing delay based on the fiducial point,
wherein the advanced pacing delay is a delay time period from delivery of a test LV septal pacing pulse of the test LV septal pacing to the fiducial point, and
wherein the fiducial point is a maximum negative QRS deflection identified within the far-field electrogram.
